(19) United States
(12) Patent Application Publication
(10) Pub. No.: US 2025/0314660 A1
(43) Pub. Date: Oct. 9, 2025

(54) METABOLIC BIOMARKERS FOR ASSESSING FRIEDREICH'S ATAXIA IN A SUBJECT

(71) Applicant: The Trustees of Indiana University, Bloomington, IN (US)

(72) Inventors: Ronald Mark Payne, Zionsville, IN (US); Thomas Michael O'Connell, Chapel Hill, NC (US)

(21) Appl. No.: 18/864,813

(22) PCT Filed: May 12, 2023
(86) PCT No.: PCT/US23/66916

§ 371 Date: Nov. 11, 2024

Related U.S. Application Data
(60) Provisional application No. 63/341,462, filed on May 13, 2022.

(51) Int. Cl. G01N 33/68; G01N 33/66; G01N 33/92

(52) U.S. Cl. CPC G01N 33/6812; G01N 33/66; G01N 33/92; G01N 2800/28; G01N 2800/52; G01N 2800/56

(57) ABSTRACT

Methods to assess efficacy of a test compound to treat Friedreich's ataxia include: determining a concentration level of a panel of at least two validated low molecular weight metabolic biomarkers in a biological sample from a subject administered the test compound; identifying a difference between the determined concentration level of the at least two metabolic biomarkers and a reference concentration level of the at least two metabolic biomarkers; and, assessing the efficacy of the test compound based on the identified difference.

### CROSS-REFERENCE TO RELATED APPLICATIONS

This application claims priority to U.S. provisional patent application No. 63/341,462 filed on May 13, 2022, and which is incorporated by reference in its entirety herein.

### FIELD

The present disclosure provides methods and systems that involve the use of metabolic biomarkers to assess Friedreich's ataxia (FA) in a subject.

### BACKGROUND

Friedreich's ataxia is an incurable genetic disorder. It is the most common inherited ataxia in humans, with an estimated 4,000-5,000 cases in the United States alone. FA is a progressive degenerative disease that affects mainly the muscular system, the nervous system, and the heart. Generally, within 10 to 15 years from onset, it leads to loss of ambulation and complete disability, with premature death often caused by cardiac insufficiency. Friedreich ataxia is caused by loss of expression of the protein frataxin, which is encoded in the nucleus and targeted to mitochondria. With loss or decreased expression of frataxin, multiple metabolic pathways are affected leading to perturbations in critical systems including cellular energy metabolism leading to cell death over time. Frataxin has no known enzymatic activity that would allow measurement of its presence or amount. Others have reported that frataxin participates in formation of mitochondrial iron-sulfur clusters, which are vital components used in many different metabolic reactions.

A major challenge for developing therapeutic drugs for FA is the inability to quickly monitor a biochemical response to a therapeutic intervention. The pharmacodynamic response to a therapeutic drug cannot be readily measured to establish a dose-response relationship. Thus, trials have used clinical endpoints, such as the modified FA Rating Scale, but power analyses and recent assessment of progression characteristics have shown that trials using these endpoints need to be conducted for at least 1 to 2 years, which is too slow for dose adjustment or toxicity assessment of a therapeutic intervention, and a need exists for a better way to more quickly monitor a biochemical response to a therapeutic intervention. The present disclosure addresses this need.

### SUMMARY

In accordance with this disclosure, sets of metabolic biomarkers can be used in a number of different ways. For patients who have been diagnoses with FA, a biomarker panel can be used to evaluate or score the severity of the disease with metabolic derangements being associated with progressively worsening disease state. In some embodiments, the biomarker panel is used as a clinical tool to provide a simple, minimally invasive (blood draw) rapid and quantitative assay to monitor the disease status.

In the context of FA therapy, a biomarker panel can be used to evaluate or score the efficacy of an intervention with the biomarker panels showing a return to or toward normal levels with effective treatment. A rapid and quantitative response to therapeutic interventions in accordance with this disclosure is also valuable in the context of a clinical trial where therapeutic efficacy, dose-response analyses and pharmacodynamics studies would all be greatly accelerated by such an assay. A biomarker panel in accordance with the disclosure can also be used to monitor a patient's response to therapy over the duration of an intervention. In some cases, positive responses to a therapeutic may have different durations in different patients and the biomarker panel can be used to monitor patients. A change in the biomarker panel away from a more normal range and toward a pre-therapy FA signature would indicate a possible need for a patient to seek a different type of treatment.

Recognizing the possibility that some of the perturbations to a panel of metabolites disclosed herein are present in FA patients prior to the observation of any other symptoms of the disease, a FA biomarker panel as disclosed herein can serve as a preliminary diagnostic panel to diagnose a patient who may be suspected of having FA. Positive results from this test could then be followed up by a definitive genetic analysis. Relatedly, this panel could be included in neonatal screening assays for inborn errors of metabolism.

In one aspect, the disclosure relates to methods to assess a subject having FA that include: (i) determining a concentration level of a panel of at least two validated low molecular weight metabolic biomarkers in a biological sample from the subject; (ii) identifying a difference between the determined concentration level of the at least two metabolic biomarkers and a reference concentration level of at least two metabolic biomarkers; and, (iii) assessing the subject based on the identified difference.

In another aspect, the disclosure relates to a method to assess efficacy of a test compound to treat FA that includes: (i) determining a concentration level of a panel of at least two validated low molecular weight metabolic biomarkers in a cell contacted with the test compound; (ii) identifying a difference between the determined concentration level of the panel of at least two metabolic biomarkers and a reference concentration level of at least two metabolic biomarkers; and, (iii) assessing the efficacy of the test compound based on the identified difference.

In another aspect, the disclosure relates to a method to assess efficacy of a test compound to treat FA that includes: (i) determining a concentration level of a panel of at least two validated low molecular weight metabolic biomarkers in a biological sample from a subject administered the test compound; (ii) identifying a difference between the determined concentration level of the panel of at least two metabolic biomarkers and a reference concentration level of at least two metabolic biomarkers; and, (iii) assessing the efficacy of the test compound based on the identified difference.

In another aspect, the disclosure relates to a diagnostic kit for assessing a subject for an attribute of FA, where the attribute of FA is associated with a low molecular weight metabolic biomarker profile. The diagnostic kit includes: a container comprising a panel of at least two validated low molecular weight metabolic biomarker internal standards having a purity greater than 98.0%, where the container is configured to receive a biological sample from the subject and to be sealed with a sealing member after receiving the biological sample.

In another aspect, the disclosure relates to a method to identify a candidate low molecular weight metabolic biomarker panel that differentially and/or comparatively diagnoses a subject having FA that includes: (i) obtaining a first biological sample from a first subject having a first phenotype associated with FA and a second biological sample from a second subject having a second phenotype associated with FA; (ii) determining a concentration level of at least two low molecular weight metabolic biomarkers in the first biological sample, the second biological sample, and a reference concentration level of the at least two metabolic biomarkers; and, (iii) identifying two or more validated low molecular weight metabolic biomarkers that are present in a statistically significant, different level in the first and second biological samples, where the different level is characterized by an area under the receiver operator characteristic (ROC) curve (AUC) ranging from 0.60 to 1.00.

In another aspect, the disclosure relates to a method to diagnose FA in a subject that includes: (i) determining a concentration level of a panel of at least two validated low molecular weight metabolic biomarkers in a biological sample from the subject; (ii) identifying a difference between the determined concentration level of the at least two metabolic biomarkers and a reference concentration level of the at least two metabolic biomarkers; and, (iii) diagnosing FA in the subject based on the identified difference.

In another aspect, the disclosure relates to a method to evaluate effectiveness of a FA treatment in a subject that has been diagnosed with FA that includes: (i) determining a concentration level of a panel of at least two validated low molecular weight metabolic biomarkers in a biological sample from the subject; (ii) identifying a difference between the determined concentration level of the at least two metabolic biomarkers and a reference concentration level of at least two metabolic biomarkers; and, (iii) assessing effectiveness of the FA treatment based on the identified difference.

In another aspect, the disclosure relates to an electronic system to assess a subject having FA. The system includes: (i) a memory; and, (ii) a processor in communication with the memory where the processor is operable to execute instructions for obtaining a concentration level of a panel of at least two validated low molecular weight metabolic biomarkers in a biological sample from the subject; identifying a difference between the determined concentration level of the at least two metabolic biomarkers and a reference concentration level of the at least two metabolic biomarkers; and, assessing the subject based on the identified difference.

In another aspect, the disclosure relates to a system to assess a subject having FA; the system including a processor operable to execute one or more computer programs, the one or more computer programs comprising instructions for carrying out a method to assess a subject having FA by: (i) identifying a difference between a determined concentration level of a panel of at least two metabolic biomarkers in a biological sample from the subject and a reference concentration level of the at least two metabolic biomarkers; and, (ii) assessing the subject based on the identified difference.

Additional embodiments, features, and advantages of the disclosure will be apparent from the following detailed description and through practice of the disclosure.

### BRIEF DESCRIPTION OF THE DRAWINGS

The teachings of some embodiments of the present disclosure will be better understood by reference to the description taken in conjunction with the accompanying drawings, wherein:

FIG. 1 shows a heatmap of significant metabolites in control and FA subjects. Metabolites are represented as z-scores with red indicating higher levels and blue indicating lower levels.

FIGS. 2A-2B show a volcano plots of Significant Metabolites (FIG. 2A) 23 hydrophilic metabolites with expansion of the three most significantly altered metabolites and (FIG. 2B) 36 lipid related metabolites with expansion of two most significantly perturbed lipid species. The red shaded region highlights the 14 TGs and two DGs which are all significantly increased except for TG_6 (TG (18:1,26:0). See Table 4, infra, for the full names of the lipid metabolites along with log₂-fold changes and p-values.

FIG. 3 shows boxplots of all significant metabolites shown in FIG. 2A. Non-standard metabolite names: C0, carnitine; Orn, ornithine; CA, cholic acid; Cit, citrulline; HCys, homocysteine; C6.1, hexenoylcarnitine; C3-OH, hydroxypropionyl-carnitine; C3.DC., C4.OH, malonylcarnitine and hydroxybutyrylcarnitine; Ind.SO4, indoxylsulfate.

FIG. 4 shows a correlogram of significant metabolite changes based on Pearson correlation coefficients. Correlations with a p-values<0.05 are shown with positive correlations in blue and negative in red. Color intensity indicates stronger correlations. Metabolites are ordered by hierarchical clustering.

FIG. 5 shows a Correlation Network Community Diagram using the Girvan-Newman algorithm to identify highly connected subgraphs representing communities of metabolites. The analysis reveals four communities indicated by the colors.

FIG. 6 shows a Correlation Network Community Diagram of the hydrophilic metabolites. The Girvan-Newman algorithm applied to a Pearson correlation matrix identify highly connected subgraphs representing communities of metabolites. The analysis reveals five communities indicated by the colors. The metabolite composition of the largest community suggests perturbations to 1C metabolism. Non-standard metabolite abbreviations include C0, carnitine; Ace, acetate; Orn, ornithine, Sarc, sarcosine; Form, formate; Suc, succinate; C3: OH, hydroxypropionlycarnitine; Hxan, hypoxanthine; HCys, homocysteine; Chol, choline and Lac, lactate.

FIG. 7 shows receiver operating characteristic curves for FA biomarkers. Legend lists the number of metabolites included in the model, the AUCs and the confidence intervals

FIG. 8 shows a diagram of specific perturbations to 1C metabolism and related pathways. The pathway diagram shows the inter-relationship of three key components of 1C metabolism including the folate cycle, purine nucleotide metabolism and methionine salvage. Metabolites in blue were altered in the serum of FA patients. Metabolite names: DMG, dimethylglycine; Gly, glycine; HCys, homocysteine; Hist, histamine; HXan, hypoxanthine; IMP, inosine monophosphate; Met, methionine; 5,10-me-THF, 5,10-methylene-THF; PCs, phosphocholines; Rib-5P, ribose-5-phosphate; Ser, serine; SAH, S-adenosylhomocysteine; SAH, S-adenosylmethionine; Sarc, sarcosine; THF, tetrahydrofolate; Xan, xanthine. *Note, uric acid was not detected by our study, but was shown to be altered by Schirinzi, et al [39]. Enzyme names: BHMT, betaine hydroxymethyltransferase; DMGDH, dimethylglycinedehydrogenase; GNMT, glycine-N-methyltransferase; HPRT1, hypoxanthine phosphoribosyltransferase 1; MTHFD, methylenetetrahydrofolate dehydrogenase; MTHFR, methylenetetrahydrofolate reductase; MTR, methionine synthase; SDH, sarcosine dehydrogenase; SHMT, serine hydroxymethyltransferase; XO, xanthine oxidase.

### DETAILED DESCRIPTION

Before the present disclosure is further described, it is to be understood that this disclosure is not limited to particular embodiments described. The embodiments described herein are not intended to be exhaustive or to limit the invention to the precise forms disclosed in the following detailed description. Rather, the embodiments are chosen and described so that others skilled in the art may appreciate and understand the principles and practices of this disclosure and, as such, may vary. It is also to be understood that the terminology used is for the purpose of describing particular embodiments only, and is not intended to be limiting, since the scope of the present disclosure will be limited only by the appended claims.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

Unless defined otherwise, all technical and scientific terms have the same meaning as is commonly understood by one of ordinary skill in the art to which this disclosure belongs. All patents, applications, published applications and other publications are incorporated by reference in their entireties. If a definition set forth in this section is contrary to or otherwise inconsistent with a definition set forth in a patent, application, or other publication that is incorporated by reference, the definition set forth in this section prevails over the definition incorporated by reference.

The singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. The terms “including,” “containing,” and “comprising” are used in their open, non-limiting sense. Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount, dose, time, temperature, enzymatic activity or other biological activity and the like, is meant to encompass variations of ±20%, ±10%, ±5%, ±1%, ±0.5%, or even ±0.1% of the specified amount. To provide a more concise description, some of the quantitative expressions are not qualified with the term “about.” It is understood that, whether the term “about” is used explicitly or not, every quantity is meant to refer to the actual given value, and it is also meant to refer to the approximation to such given value that would reasonably be inferred based on the ordinary skill in the art, including equivalents and approximations due to the experimental and/or measurement conditions for such given value.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. It should be further appreciated that although reference to a “preferred” component or feature may indicate the desirability of a particular component or feature with respect to an embodiment, the disclosure is not so limiting with respect to other embodiments, which may omit such a component or feature. Further, when a particular feature, structure, or characteristic is described in connection with an embodiment, it is submitted that it is within the knowledge of one skilled in the art to implement such feature, structure, or characteristic in connection with other embodiments whether or not explicitly described. Additionally, it should be appreciated that items included in a list in the form of “at least one of A, B, and C” can mean (A); (B); (C); (A and B); (B and C); (A and C); or (A, B, and C). Similarly, items listed in the form of “at least one of A, B, or C” can mean (A); (B); (C); (A and B); (B and C); (A and C); or (A, B, and C). Further, with respect to the claims, the use of words and phrases such as “a,” “an,” “at least one,” and/or “at least one portion” should not be interpreted so as to be limiting to only one such element unless specifically stated to the contrary, and the use of phrases such as “at least a portion” and/or “a portion” should be interpreted as encompassing both embodiments including only a portion of such element and embodiments including the entirety of such element unless specifically stated to the contrary. All combinations of the embodiments pertaining to the metabolic biomarkers represented by the variables are specifically embraced by the present disclosure just as if each and every combination was individually and explicitly disclosed. In addition, all subcombinations of the chemical groups listed in the embodiments describing such variables are also specifically embraced by the present disclosure just as if each and every such sub-combination of metabolic biomarkers was individually and explicitly disclosed.

Before the present disclosure is further described, it is to be understood that this disclosure is not limited to particular embodiments described, as such may, of course, vary. It is also to be understood that the terminology used is for the purpose of describing particular embodiments only, and is not intended to be limiting, since the scope of the present disclosure will be limited only by the appended claims.

Unless defined otherwise, all technical and scientific terms used have the same meaning as is commonly understood by one of ordinary skill in the art to which this disclosure belongs. All patents, applications, published applications and other publications referred to are incorporated by reference in their entireties. If a definition set forth in this section is contrary to or otherwise inconsistent with a definition set forth in a patent, application, or other publication that is incorporated by reference, the definition set forth in this section prevails over the definition incorporated by reference.

### Definitions

“Asymptomatic” or “pre-symptomatic” refer to a subject with FA as defined by a diagnosis but with no overt clinical neurological, musculoskeltal or cardiac phenotype.

“Early-symptomatic” refers to a subject with FA as defined by a diagnosis and with the presence of mild clinical neurological or cardiac phenotype.

“Symptomatic” or “post-symptomatic” refers to a subject with FA as defined by a diagnosis and with the presence of neurological phenotypes (sensory and spinocerebellar ataxia, dysarthria, vision and hearing loss, dysphagia, areflexia, loss of joint position and vibration sense in the lower limbs), or cardiac phenotypes (hypertrophic cardiomyopathy, dilated cardiomyopathy, arrhythmias, heart failure).

“Late-symptomatic” refers to a subject with FA as defined by a diagnosis and with the presence of symptomatic findings that have advanced to the point that the subject is wheelchair bound, incapable of controlled voluntary movements, must be cared for and fed by family members or attendants continuously, and may/may not have severe heart failure.

“Ataxia” refers to both cerebellar ataxia and spinal ataxia (including posterior spinal ataxia), and generally involves the loss or failure of muscular coordination. Subjects exhibiting ataxia may have difficulty regulating the force, range, direction, velocity, and rhythm of muscles involved in posture and balance. Ataxia of the trunk muscles, for example, can result in increased postural sway, and an inability to maintain the center of gravity over the base of support. Ataxia and primary or secondary symptoms of ataxic gait and tremor of the extremities may also lead to manifestations of speech disturbance, dysphagia, abnormal ventilation, and involuntary movements such as dystonia, and sometimes develops into vegetative symptoms or spastic paraplegia, as well as pyramidal or extrapyramidal symptoms, thereby substantially interfering with the activities of daily life.

“Heart failure” refers to both dilated and hypertrophic heart failure in which the function of the heart has been compromised and cannot meet the metabolic demands of the body. The heart may also develop arrhythmias that compromise cardiac function and may lead to heart failure and/or death or stroke. The heart in FA may develop either failure of systolic function, or diastolic function, and either may lead to the clinical diagnosis of heart failure. The presence of heart failure may significantly contribute to, or cause, inability to participate in normal daily activities of life, such as walking, normal digestion of food and it's elimination, and metabolic disruption leading to death or stroke.

“Energy metabolism biomarker” refers to a metabolite that participates in one of the canonical energy generating pathways, e.g., glycolysis, the TCA cycle, fatty acid oxidation or that is associated with the gut microbiome. Examples include, but are not limited to, acetate, glucose, lactate and pyruvate, citrate, succinate, fumarate, malate, carnitine and acylcarnitines (i.e. carnitine conjugated with a range of fatty acids with carbon chain lengths ranging from 2 to 18 with varying degrees of unsaturation and potential modifications by hydroxylation, carboxylation and dicarboxylation).

“Amino acid metabolism biomarker” refers to an amino acid that plays a number of roles including biosynthesis of protein, energy metabolism, and a range of cell signaling. “Amino acid” refers to the 20 naturally occurring canonical amino acids which are natural components of proteins as well as the non-canonical amino acids which includes non-proteogenic amino acids that are found naturally in organisms or made synthetically. There are 20 naturally occurring amino acids encoded by the genetic code. Amino acids can be referred to by either their commonly known three-letter symbols or by the one-letter symbols recommended by the IUPAC-IUB Biochemical Nomenclature Commission. Exemplary low molecular weight naturally occurring amino acid metabolic biomarkers include, but are not limited to:

| Amino Acid | Abbreviation |
|---|---|
| Alanine | Ala |
| Arginine | Arg |
| Leucine | Leu |
| Lysine | Lys |
| Asparagine | Asn |
| Aspartate | Asp |
| Methionine | Met |
| Phenylalanine | Phe |
| Cysteine | Cys |
| Glutamate | Glu |
| Proline | Pro |
| Serine | Ser |
| Glutamine | Gln |
| Glycine | Gly |
| Threonine | Thr |
| Tryptophan | Trp |
| Histidine | His |
| Isoleucine | Ile |
| Tyrosine | Tyr |
| Valine | Val |

Furthermore, there are a number of non-canonical amino acids that meet the definition of an amino acid, possessing both an amino moiety and an acid moiety, but are not part of the set of 20 canonical amino acids. These non-natural amino acids have a wide range of functions including energy metabolism, cellular stress response, ammonia metabolism and inflammatory and immune responses. Examples of low molecular weight non-canonical amino acid metabolic biomarkers include, but are not limited to:

Non-canonical amino acids

1-Methylhistidine
3-Methylhistidine
5-Aminovaleric acid
α-Aminoadipic acid
α-Aminobutyric acid
β-Aminobutyric acid
β-Aminoisobutyric acid
γ-Aminobutyric acid
Acetylornithine
Anserine
Asymmetric dimethylarginine
Betaine
Carnosine
cis-4-hydroxyproline
Citrulline
Creatine
Creatinine
Cystine
Dihydroxyphenylalanine
Homoarginine
Homocysteine
Kynurenine
Methionine sulfoxide
Nitrotyrosine
Ornithine
Phenylacetylglycine
Phenylalanin betaine
Proline betaine
Sarcosine
Symmetric dimethylargine
Taurine
trans-4-Hydroxyproline
Tryptophan betaine

“Fatty acid metabolism biomarker” refers to a metabolite that include the acylcarnitines which participate in fatty acid oxidation in the β- and ω-oxidation pathways. Other metabolites include longer chain, i.e., 12 carbons or longer, free fatty acids including saturated and unsaturated. “Acylcarnitine” refers to fatty acyl esters of L-carnitine. “Carnitine” is an amino acid derivative and nutrient involved in lipid metabolism in mammals and other eukaryotes. Carnitine is in the chemical compound classes of β-hydroxyacids and quaternary ammonium compounds, and because of the hydroxyl-substituent, it exists in two stereoisomers: the biologically active enantiomer L-carnitine, and the essentially biologically inactive D-carnitine. Carnitine participates in the carnitine shuttle system which functions to transport fatty acids across the mitochondrial membrane for subsequent catabolismacylcarnitines (AC) are formed from a family of carnitine acyltransferases that exchange a CoA group for a carnitine. AcylCoA species cannot cross the mitochondrial membrane, but the ACs can. Once inside the mitochondria, these transferases can shuttle the ACs out of the mitochondria into the circulation. Serum ACs are thus a useful metabolic surrogate for intermediates along the β-oxidation pathway. Exemplary low molecular weight acylcarnitine metabolic biomarkers include, but are not limited to:

Acylcarnitine Species

| Abbreviation | Common Name |
|---|---|
| C0 | Carnitine |
| C2 | Acetylcarnitine |
| C3 | Propionylcarnitine |
| C3-DC | Malonylcarnitine |
| C4-OH | Hydroxybutyrylcarnitine |
| C3-OH | Hydroxypropionylcarnitine |
| C3:1 | Propenoylcarnitine |
| C4 | Butyrylcarnitine |
| C4:1 | Butenylcarnitine |
| C5 | Valerylcarnitine |
| C5-DC | Glutarylcarnitine |
| C6-OH | Hydroxyhexanoylcarnitine |
| C5-M-DC | Methylglutarylcarnitine |
| C5-OH | Hydroxyvalerylcarnitine |
| C3-DC-M | Methylmalonylcarnitine |
| C5:1 | Tiglylcarnitine |
| C5:1-DC | Glutaconylcarnitine |
| C6 | Hexanoylcarnitine |
| C4:1-DC | Fumarylcarnitine |
| C6:1 | Hexenoylcarnitine |
| C7-DC | Pimeloylcarnitine |
| C8 | Octanoylcarnitine |
| C9 | Nonaylcarnitine |
| C10 | Decanoylcarnitine |
| C10:1 | Decenoylcarnitine |
| C10:2 | Decadienoylcarnitine |
| C12 | Dodecanoylcarnitine |
| C12-DC | Dodecanedioylcarnitine |
| C12:1 | Dodecenoylcarnitine |
| C14 | Tetradecanoylcarnitine |
| C14:1 | Tetradecenoylcarnitine |
| C14:1-OH | Hydroxytetradecenoylcarnitine |
| C14:2 | Tetradecadienoylcarnitine |
| C14:2-OH | Hydroxytetradecadienoylcarnitine |
| C16 | Hexadecanoylcarnitine |
| C16-OH | Hydroxyhexadecanoylcarnitine |
| C16:1 | Hexadecenoylcarnitine |
| C16:1-OH | Hydroxyhexadecenoylcarnitine |
| C16:2 | Hexadecadienoylcarnitine |
| C16:2-OH | Hydroxyhexadecadienoylcarnitine |
| C18 | Octadecanoylcarnitine |
| C18:1 | Octadecenoylcarnitine |
| C18:1-OH | Hydroxyoctadecenoylcarnitine |
| C18:2 | Octadecadienylcarnitine |

Other exemplary fatty acid metabolism biomarkers include, but are not limited to FA 20.3 and dodecanedioic acid.

“Urea cycle biomarker” refers to a metabolite that plays a role in the urea cycle, also known as the ornithine cycle which is involved in the detoxification of ammonia. Examples include, but are not limited to arginine, citrulline and ornithine.

Urea Cycle Related Metabolites
Common Name

Glutamate
N-acetylglutamate
citrulline
ornithine
asparate
arginosuccinate
urea
orotic acid

“One-carbon metabolism biomarker” refers to a metabolite that plays a role in a one carbon metabolism related pathway. One carbon metabolism related pathways include the folate cycle, methionine salvage and purine nucleotide metabolism and play a role in the generation of methyl groups which are used in methylation reactions that modulate a wide range of cellular processes. Examples include, but are not limited to, formate, homocysteine, hypoxanthine, choline, sarcosine, histamine, glycine, methionine, dimethylglycine, serine, spermine, spermidine, putrescine, aspartate, S-adenosylmethionine, S-adenosylhomocysteine, inosine monophosphate, uric acid, tetrahydrofolate, 5,10-methylenetetrahydrofolate, 10-formyltetrahydrofolate, 5-methyltetrahydrofolate, and adenine.

“Cholesterol metabolism biomarker” refers to a bile acid or a cholesterol ester, such as, for example and without limitation, cholesterol ester 20:0 (CE.20.0) that plays a role in the solubilization of lipids and cholesterol. “Bile acid” refers to metabolites that promote fat absorption by acting as potent “digestive surfactants” to lipids (including fat-soluble vitamins) by acting as emulsifiers. Bile acids constitute a large family of molecules, composed of a steroid structure with four rings, a five or eight carbon side-chain terminating in a carboxylic acid, and the presence and orientation of different numbers of hydroxyl groups. The four rings are labeled from left to right on Bile Acid Formula, shown below as A, B, C, and D, with the D-ring being smaller by one carbon than the other three. The hydroxyl groups have a choice of being in 2 positions, beta (solid pie-shaped line), or alpha (dashed line). All bile acids have a hydroxyl group on position 3, which was derived from the parent molecule, cholesterol. In cholesterol, the 4 steroid rings are flat and the position of the 3-hydroxyl is beta.

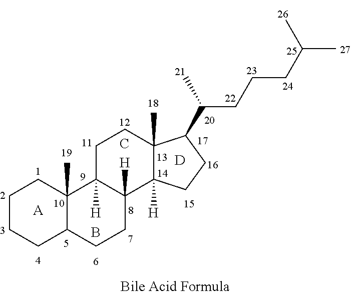

The immediate products of the bile acid synthetic pathways are referred to as primary bile acids. Cholic acid and chenodeoxycholic acid (shown below) are two forms of primary bile acids formed in humans. The action of intestinal bacterial flora on primary bile acids results in the formation of secondary bile acid species: deoxycholic, lithocholic, and ursodeoxycholic acid. Deoxycholic acid is derived from cholic acid and lithocholic acid and ursodeoxycholic acid are derived from chenodeoxycholic acid. Exemplary low molecular weight bile acid biomarkers are known to those skilled in the art.

Much of the secreted bile acids are in the form of conjugates with the amino acids taurine or glycine and/or conjugates with sulfate. The terms “conjugating,” “conjugation” and “conjugated” refer to the formation of a covalent bond. Conjugation of bile acids are catalyzed by enzymatic reactions that convert the bile acid to an acyl-CoA thioester then transfer the bile acid moiety from the acyl-CoA thioester to either glycine or taurine to form the respective bile acid conjugate. These additions substantially increase the acidity of the molecules and their solubility in water. At the physiological pH values in the intestines, the bile acid conjugates ionize and exist in salt form. In the conjugated state, the molecules cannot passively enter the epithelial cells of the biliary tract and small intestines.

“Bile acid,” includes bile acid alcohols, sterols, and salts thereof, found in the bile of an animal (e.g., a human), including, by way of non-limiting example, cholic acid, cholate, deoxycholic acid, deoxycholate, hyodeoxycholic acid, hyodeoxycholate, glycocholic acid, glycocholate, taurocholic acid, taurocholate and the like. Taurocholic acid and/or taurocholate are referred to as TCA. Any reference to a bile acid includes reference to a bile acid, one and only one bile acid, one or more bile acids, or to at least one bile acid. Therefore, the phrases “bile acid,” “bile salt,” “bile acid/salt,” “bile acids,” “bile salts,” and “bile acids/salts” are, unless otherwise indicated, utilized interchangeably. Any reference to a bile acid includes reference to a bile acid or a salt thereof. Furthermore, “bile acids” include bile acids conjugated to an amino acid (e.g., glycine or taurine). For example, the phrase “bile acid” includes cholic acid conjugated with either glycine or taurine: glycocholate and taurocholate, respectively (and salts thereof). Furthermore, it is to be understood that any singular reference to a component (bile acid or otherwise) includes reference to one and only one, one or more, or at least one of such components. Similarly, any plural reference to a component includes reference to one and only one, one or more, or at least one of such components, unless otherwise noted. Examples of low molecular weight bile acid metabolic biomarkers include, but are not limited to:

| Bile Acid | Type |
|---|---|
| Cholic acid | primary |
| Chenodeoxycholic acid | primary |
| α-Muricholic acid | primary |
| β-Muricholic acid | primary |
| ω-Murichoclic acid | primary |
| Deoxycholic acid | secondary |
| Lithocholic acid | secondary |
| Hyodeoxycholic acid | secondary |
| Ursodeoxycholic acid | secondary |
| Glycocholic acid | conjugated primary |
| Glycochenodeoxycholic acid | conjugated primary |
| Taurocholic acid | conjugated primary |
| Taurochenodeoxycholic acid | conjugated primary |
| Tauromuricholic acid | conjugated primary |
| Glycodeoxycholic acid | conjugated secondary |
| Glycolithocholic acid | conjugated secondary |
| Taurodeoxycholic acid | conjugated secondary |
| Taurolithocholic acid | conjugated secondary |
| Glycoursodeoxycholic acid | conjugated secondary |
| Tauroursodeoxycholic acid | conjugated secondary |

“Phospholipid metabolism biomarker” refers to a phospholipid including a range of glycerophospholipids with 2 fatty acid side chains linked by ester (designated with aa) or ether (designated with ae) bonds. These lipids play a role in membrane synthesis as well as a diverse range of signaling functions. These lipids also contain a sphingomyelin and ceramide species which play similar roles. Examples include, but are not limited to, PC.aaC26.0, PC.aaC30.0, PC.aaC32.0, PC.aaC34.2, PC.aaC36.2, PC.aaC40.1, PC.aaC40.2, PC.aeC34.1, PC.aeC34.2, PC.aeC34.3, PC.aeC36.2, PC.aeC36.3, PC.aeC36.4, PC.ae42.4, PC.aeC44.4 and SM.C24.0.

“Fatty acid storage/transport biomarker” refers to a triacylglycerol found in adipose tissue stores and in lipoproteins. They are stored during periods of nutrient excess and mobilized for energy when needed. They circulate as components of lipoproteins. The metabolites contain three fatty acid side chains with varying lengths and degrees of unsaturation. The named metabolites are described by the chain length and degrees of unsaturation of one chain followed by the sum of the chain lengths and degrees of unsaturation of the other two. This pathway also contains diacylglycerols which are hydrolysis products of TAGs where one fatty acid side chain has been hydrolyzed off. Examples include, but are not limited to, Cer.d18.0.22.0, DG.16.1_18.1, DG.16.1_18.2, TG.16.0_28.2, TG.16.0_38.4, TG.20.0_32.4, TG.17.1_36.3, TG.17.1_36.4, TG.18.1_34.3, TG.18.1_36.4, TG.18.1_36.5, TG.18.2_36.3, TG.18.3_34.2, TG.18.3_36.2, TG.20.0_32.3, and TG.20.0_32.4.

“Lipoprotein metabolism biomarker” refers to a member of a group that contains the lipoprotein particle subfraction numbers and sizes determined by nuclear magnetic resonance. Lipoproteins are involved in the transportation of fatty acid and cholesterol throughout the body including intestines, liver, skeletal muscle, heart and adipose tissue. Examples of lipoprotein metabolism biomarkers include, but are not limited to:

| Lipoprotein Particle Name | ID |
|---|---|
| VLDL & Chylomicron Particles (total) | VLDLCP3 |
| Large VLDL & Chylomicron Particles | VLCP3 |
| Medium VLDL Particles | VMP3 |
| Small VLDL Particles | VSP3 |
| LDL Particles (total) | LDLP3 |
| IDL Particles | IDLP3 |
| Large LDL Particles | LLP3 |
| Small LDL Particles (total) | LSP3 |
| HDL Particles (total) | HDLP3 |
| Large HDL Particles | HLP3 |
| Medium HDL Particles | HMP3 |
| Small HDL Particles | HSP3 |
| VLDL Size | VZ3 |
| LDL Size | LZ3 |
| HDL Size | HZ3 |
| GlycA | GLYCA |
| Total Cholesterol | ELP_TC |
| HDL Cholesterol | ELP_HDLC |
| Triglycerides | ELP_TG |
| ApoB | ELP_APOB |
| LDL Cholesterol | ELP_LDLC |
| VLDL Cholesterol | ELP_VLDLC |
| Non-HDL Cholesterol | ELP_NONHDLC |

“Gut microbial metabolism biomarker” refers to compounds that are the result of a metabolic interaction between the host and the particular set of gut microbes found in the GI tract of the patient. Such biomarkers can be derived from the diet whereby dietary compounds are metabolized by the microbes and subsequently enter the patient's circulation. Metabolites from the host i.e. the patient can also enter the gut via enterohepatic circulation and thus host-derived compounds can be metabolized by the microbes and then re-enter the host circulation. Examples of compounds that are the result of gut microbial metabolism include but are not limited to the following.

Common Gut Microbiome Related Metabolites

| Common Name | Description |
|---|---|
| acetate | short chain fatty acid |
| propionate | short chain fatty acid |
| butyrate | short chain fatty acid |
| trimethylamine-N-oxide | choline/carnitine metabolite |
| indoxyl sulfate | tryptophan metabolite |
| deoxycholic acid | secondary bile acid |
| glycocholic acid | secondary bile acid |
| glycochenodeoxycholic acid | secondary bile acid |

“Area under curve” or “AUC” refers to area under a receiver operating characteristic (ROC) curve. AUC under a ROC curve is a measure of the predictive accuracy of a model. An area of 1 represents a perfect test, whereas an area of 0.5 represents a test that is no better than random guesses. A preferred AUC may be at least approximately 0.700, at least approximately 0.750, at least approximately 0.800, at least approximately 0.850, at least approximately 0.900.

“Assess” or “assessing” refers to any form of measurement and includes determining if an element is present or absent and both quantitative and qualitative determination in the sense of obtaining an absolute value for the amount or concentration of the metabolic biomarker or metabolic biomarkers to be analyzed present in the sample, and also obtaining an index, ratio, percentage or other value indicative of the level of metabolic biomarker in the sample. Assessment may be direct or indirect and the chemical species actually detected need not of course be the analyte itself but may for example be a derivative thereof. The purpose of such assessment of metabolic biomarkers may be different.

“Biological sample” refers to any biological sample obtained from a subject or a group or population of subjects that may contain relevant metabolites, including tissue and biological fluids such as, but not limited to, blood, urine, sweat, saliva, and sputum. The biological sample is obtained from the subject in a manner well-established in the art. “Blood” as used herein encompasses whole blood, blood plasma, and blood serum. The biological sample, like blood samples, may be analyzed without or after a pre-treatment. Examples of pre-treated blood samples are pre-treated blood, like EDTA-blood, or EDTA-plasma, citrate-plasma, heparin plasma. The originally obtained (blood) samples or fractions thereof may be further modified by methods known in the art, as for example by filtration, ultrafiltration, fractionation, and/or dilution. Fractionation may be performed to remove constituents that might disturb the analysis. Dilution may be performed by mixing the original (blood) sample or fraction with a suitable sample liquid, like a suitable buffer with or without water and/or deuterated water (D20), to adjust the concentration the constituents, as for example of the analyte. Such modified (blood) samples exemplify samples “derived from” the original body fluid sample collected or isolated from the body of the individual.

“Chromatography” refers to a physical method of separation in which the components to be separated are distributed between two phases, one of which is stationary (stationary phase) while the other (the mobile phase) moves in a definite direction. The mobile phases can be aqueous or organic solvents, or mixtures thereof, as used in liquid chromatography or gasses such as helium, nitrogen or argon as used in gas chromatography. Chromatographic output data may be used for manipulation by the present disclosure.

“Determining” refers to methods that include identifying the presence or absence of metabolic biomarkers in the sample, quantifying the amount of substance(s) in the sample if present, and/or qualifying the type of substance. “Determining” likewise refers to methods that include identifying the presence or absence of specific metabolic biomarkers. A “positive” reference concentration level of a metabolic biomarker means a level that is indicative of a particular FA state or phenotype. A “negative” reference concentration level of a metabolite means a level that is indicative of a lack of a particular FA state or phenotype. For example, a “heart failure-positive reference level” of a metabolic biomarker means a level of a metabolite that is indicative of a positive diagnosis of heart failure in a FA subject, and a “heart failure-negative reference level” of a metabolite means a level of a metabolite that is indicative of a negative diagnosis of heart failure in a FA subject. A “reference concentration level” of a metabolite may be an absolute or relative amount or concentration of the metabolite, a presence or absence of the metabolite, a range of amount or concentration of the metabolite, a minimum and/or maximum amount or concentration of the metabolite, a mean amount or concentration of the metabolite, and/or a median amount or concentration of the metabolite; and, in addition, “reference levels” of combinations of metabolites may also be ratios of absolute or relative amounts or concentrations of two or more metabolites with respect to each other or a composed value/score obtained by a statistical model.

“Effective amount” is that amount sufficient, at dosages and for periods of time necessary, to achieve a desired therapeutic result, such as for treatment of a FA phenotype, a condition, and/or pharmacokinetic or pharmacodynamic effect of the treatment in a subject. The therapeutically effective amount may vary according to factors such as the disease state, age, sex, and weight of the subject. In some embodiments, the present disclosure may be used to determine an effective amount of a test compound.

“Friedreich's ataxia” refers to a rare, inherited autosomal recessive mitochondrial metabolism disorder that results in a progressive neuro- and cardio-degenerative disease and typically begins in childhood. It is caused by the low expression of a small mitochondrial protein, frataxin (FXN), and leads to severe disruption of mitochondrial metabolism, loss of motor skills and a cardiomyopathy within 10-15 years of diagnosis. Subjects develop a primary neurodegeneration of the dorsal root ganglia (DRG) leading to the hallmark clinical findings of progressive ataxia and debilitating scoliosis. A significant percentage of subjects will also develop a severe hypertrophic cardiomyopathy leading to death in the 3rd to 5th decade of life.

“In vitro” refers to events that occur in an artificial environment, e.g., in a test tube or reaction vessel, in cell culture, etc., rather than within a multi-cellular organism.

“In vivo” refers to events that occur within a multi-cellular organism, such as a human and a non-human animal. In the context of cell-based systems, the term may be used to refer to events that occur within a living cell (as opposed to, for example, in vitro systems).

“Metabolite” refers to any compound produced or used during all the physical and chemical processes within the body that create and use energy or are involved in biosynthetic or catabolic processes which maintain the healthy homeostatic state of the organism. Such processes include digesting food and nutrients, eliminating waste through, breathing, circulating blood, and regulating temperature, mounting inflammatory and immune responses, etc. Metabolites also refer to compounds produced through the action of the gut microbiota.

“Level” refers to a quantifiable amount of a metabolite in a sample. For example, the level may be a concentration level from a chromatography, nuclear magnetic resonance (NMR) spectroscopy or mass spectrometry comprising analysis for a metabolite. The level may be the concentration derived from specific chemical assays and/or Enzyme Linked Immunosorbent Assay (ELISA). ELISA is an assay in which a ligand, such as a metabolite, binds to a protein and the ligand-protein complex is detected by an antibody. This then generates a signal that can be used to quantify the concentration of the ligand. The level of the metabolite in a sample may be expressed as arbitrary units related to concentration or as actual concentration units such as millimolar (mM), micromolar (μM), nanomolar (nM), or picomolar (pM). The data may also be reported as ratios of metabolites or sets of metabolites. These data may be calculated from data from an assay and may be based on calibration data. A “different level” or “elevated level” of a metabolite refers to the amount or concentration of a metabolite in a sample from a subject compared to statistically validated thresholds, e.g., the amount of the metabolite in a sample(s) from individual(s) that do not have FA, have FA (or a particular severity or stage of FA), have no symptoms of a phenotype associated with FA, or have one or more phenotypes but do not have others. A “different level” or “elevated level” of a metabolite also may refer to the amount or concentration of a metabolite in a sample from a subject compared to statistically validated thresholds. “Change” in the level of at least two metabolic biomarkers refers to an increase or a decrease of by about 1.2-fold or greater in the level of the metabolic biomarkers as determined in a biological sample obtained from the subject as compared to the reference level of the at least two metabolic biomarkers. In one embodiment, the change in level is an increase or decrease by about 1.2-fold. Fold change is calculated as (New value)/(Old value).

“Low molecular weight metabolic biomarker” refers to endogenous organic compounds of a cell, an organism, a tissue or being present in body liquids, in particular blood, and in extracts or fractions obtained from blood such as plasma. Typical examples of metabolites are compounds from chemical classes including organic acids (carboxylic acids), carbohydrates (hexoses), organic amines (α-hydroxy acids, α-keto acids, β-hydroxy amino acids, acylcarnitines, trimehylamines), fatty acids (unsaturated fatty acids, dicarboxylic acids), amino acids (non-essential, essential, non-proteogenic, sulfur amino acids, secondary amines), organic sulfuric acids, purines (oxypurines), alkylamines, bile acids (primary bile acids), cholesterol esters (fatty acid esters), lipids (glycerophosphocholines, sphingomyelins, ceramides, diacylglycerols, triglycerides), lipoproteins (LDLs, HDLs) and other compounds known to those skilled in the art. This includes any substance produced by metabolism or by a metabolic process and any substance involved in metabolism. In particular, suitable metabolites are described in Table A. More particular, they may have a molecular weight typically of up to 1500 Dalton, as for example in the range of 50 to 1500 Dalton. A “metabolite” is an intermediate or product resulting from metabolism. Metabolites are often referred to as “small molecules.” Metabolites are molecules produced through metabolism in the body of a specified compound or salt thereof. Metabolites may be identified and quantified using analytical techniques known to those skilled in the art. “Biomarker” refers to concentration data of at least one, as for example 2, 3, 4, 5, 6, 7, 8, 9 or 10, 11, 12, 13, 14, 15, 16, 17, 18, 19, 20, 21, 22, 23, 24, 25, and more than 25 metabolites (also designated as a “panel” of metabolites, “signature” of metabolites, “model” or “profile” using quantitative data or concentration data directly or processed by any mathematical transformation (e.g., by log transformation, unit variance scaling, Pareto scaling or a classification method) and evaluated as an indicator of biologic processes or responses to a therapeutic intervention. In embodiments, the metabolite panel may be a linear combination of metabolite concentrations determined by a statistical procedure such that each metabolite has a coefficient that provides mathematical weight to the contributions of each metabolite to the overall panel. “Biomarker” is intended to also comprise ratios between two or more metabolites/biomarkers. Thus, the term “biomarker” may also encompass the ratio of the amount of two or more metabolites. A low molecular weight metabolic biomarker is not a protein. Examples of low molecular weight metabolic biomarkers are set forth in Table 1.

TABLE 1

| | Chemical | Chemical | Pathway | | |
|---|---|---|---|---|---|
| Metabolite | Class (1) | Class (2) | (1) | P-value | Log₂FC |
| Cholesterol ester | cholesterol | fatty acid | cholesterol | 3.97E−05 | −0.4244645 |
| 20:0 (CE.20.0) | ester | ester | metab | | |
| Sarcosine | amino | secondary | 1-carbon | 0.000107735 | −1.84145182 |
| | acid | amine | metab | | |
| Formate | organic | carboxylic | 1-carbon | 0.000122276 | −1.17012103 |
| | acid | acid | metab | | |
| ELP_HDLC | lipoprotein | HDL | lipoprotein | 0.000354119 | −0.82698838 |
| | | | metab | | |
| Hypoxanthine | purine | oxopurine | 1-carbon | 0.000379909 | −1.65041123 |
| | | | metab | | |
| Cer.d18.0.22.0. | lipid | ceramide | fatty acid | 0.000506326 | −2.2432408 |
| | | | transport | | |
| Glucose | carbohydrate | hexose | energy | 0.000550018 | 0.339565708 |
| | | | metab | | |
| HLP3 | lipoprotein | HDL | lipoprotein | 0.000625974 | −1.1919428 |
| | | | metab | | |
| Ornithine (Orn) | amino acid | non protegenic | urea cycle | 0.001720807 | −0.82846177 |
| LZ3 | lipoprotein | LDL | lipoprotein | 0.001902605 | NA |
| | | | metab | | |
| Dodecanedioic | fatty | Dicarboxylic | fatty acid | 0.002058313 | 0.371143752 |
| acid (DiCA.12.0) | acid | acid | metab | | |
| HDLP3 | lipoprotein | HDL | lipoprotein | 0.002286259 | −0.47278858 |
| | | | metab | | |
| PC.ae.C36.3 | lipid | Glycerophos- | phospholipid | 0.002761428 | −0.51869087 |
| | | phocholine | metab | | |
| Arg | amino acid | non-essential | urea cycle | 0.003651663 | 0.576977859 |
| PC.ae.C36.2 | lipid | Glycerophos- | phospholipid | 0.004837951 | −0.4320204 |
| | | phocholine | metab | | |
| PC.ae.C34.2 | lipid | Glycerophos- | phospholipid | 0.005365672 | −0.59460058 |
| | | phocholine | metab | | |
| Lactate | organic | a-hydroxy | energy | 0.00619195 | −0.51820009 |
| | acid | acid | metab | | |
| Hydroxypropionyl- | organic | Acylcarnitine | fatty acid | 0.006459497 | −0.23666169 |
| carnitine | acid/amine | | metab | | |
| (C3•OH) | | | | | |
| PC.ae.C34.3 | lipid | Glycerophos- | phospholipid | 0.007949739 | −0.56231064 |
| | | phocholine | metab | | |
| Choline | organic | trimethylamine | 1-carbon | 0.009129801 | −0.36151298 |
| | amine | | metab | | |
| LLP3 | lipoprotein | LDL | lipoprotein | 0.009692784 | −1.58364597 |
| | | | metab | | |
| PC.aa.C40.2 | lipid | Glycerophos- | phospholipid | 0.012396677 | −0.4636444 |
| | | phocholine | metab | | |
| carnitine (C0) | organic | b-hydroxy | fatty acid | 0.012718448 | 0.427506962 |
| | acid/amine | amino acid | metab | | |
| Thr | amino acid | essential | amino acid | 0.01368503 | −0.43714639 |
| | | | metab | | |
| TG.18.3_36.2. | lipid | Triglyceride | fatty acid | 0.015871126 | 0.841731288 |
| | | | transport | | |
| LSP3 | lipoprotein | LDL | lipoprotein | 0.015896642 | 1.074535012 |
| | | | metab | | |
| Hexenoylcarnitine | organic | Acylcarnitine | fatty acid | 0.016304846 | −0.25751608 |
| (C6.1) | acid/amine | | metab | | |
| DG.16.1_18.2. | lipid | Diacylglycerol | fatty acid | 0.019664546 | 0.388355745 |
| | | | transport | | |
| PC.aa.C40.1 | lipid | Glycerophos- | phospholipid | 0.020096311 | −0.32490141 |
| | | phocholine | metab | | |
| HZ3 | lipoprotein | HDL | lipoprotein | 0.024036423 | −0.07192209 |
| | | | metab | | |
| SM.C24.0 | lipid | Sphingomyelin | phospholipid | 0.02418962 | −0.41340562 |
| | | | metab | | |
| Acetate | organic | carboxylic | energy | 0.026396585 | 0.447204111 |
| | acid | acid | metab | | |
| TG.18.1_36.5. | lipid | Triglyceride | fatty acid | 0.026445595 | 0.91735929 |
| | | | transport | | |
| PC.ae.C44.4 | lipid | Glycerophos- | phospholipid | 0.026494636 | −0.2041944 |
| | | phocholine | metab | | |
| Histamine | alkylamine | deg of His | 1-carbon | 0.026619814 | −0.05381249 |
| | | | metab | | |
| TG.16.1 36.3. | lipid | Triglyceride | fatty acid | 0.028934841 | 0.667284342 |
| | | | transport | | |
| FA.20.3. | fatty | unsaturated | fatty acid | 0.028986995 | −0.73330215 |
| | acid | fatty acid | metab | | |
| PC.aa.C36.2 | lipid | Glycerophos- | phospholipid | 0.029479808 | −0.2340648 |
| | | phocholine | metab | | |
| TG.16.0_28.2. | lipid | Triglyceride | fatty acid | 0.029479808 | 0.067621448 |
| | | | transport | | |
| TG.18.1 26.0. | lipid | Triglyceride | fatty acid | 0.029479808 | −0.10451538 |
| | | | transport | | |
| TG.20.0_32.3. | lipid | Triglyceride | fatty acid | 0.029479808 | 0.426180411 |
| | | | transport | | |
| malonylcarnitine | organic | Acylcarnitine | fatty acid | 0.031457228 | −0.41934827 |
| (C3•DC) | acid/amine | | metab | | |
| Hydroxybutyryl- | organic | Acylcarnitine | fatty acid | 0.031457228 | −0.41934827 |
| carnitine | acid/amine | | metab | | |
| (C4•OH) | | | | | |
| Lys | amino | essential | amino acid | 0.0316232 | −0.35375046 |
| | acid | | metab | | |
| PC.aa.C34.2 | lipid | Glycerophos- | phospholipid | 0.0316232 | −0.21464041 |
| | | phocholine | metab | | |
| Citrulline (Cit) | amino | non protegenic | urea cycle | 0.031678582 | −0.31865575 |
| | acid | | | | |
| PC.aa.C26.0 | lipid | Glycerophos- | phospholipid | 0.031678582 | −0.5187894 |
| | | phocholine | metab | | |
| PC.ae.C36.4 | lipid | Glycerophos- | phospholipid | 0.031678582 | −0.4747879 |
| | | phocholine | metab | | |
| Cholic | bile | primary | bile acid | 0.034464306 | −0.65853141 |
| Acid (CA) | acid | bile acid | metab | | |
| TG.18.3_34.2. | lipid | Triglyceride | fatty acid | 0.03458172 | 0.793912577 |
| | | | transport | | |
| Leu | amino | essential | amino acid | 0.03570011 | −0.28398447 |
| | acid | | metab | | |
| Homocysteine | amino | sulfur | 1-carbon | 0.03570011 | −0.54803616 |
| (Hcys) | acid | amino acid | metab | | |
| PC.aa.C32.0 | lipid | Glycerophos- | phospholipid | 0.041008324 | −0.30076552 |
| | | phocholine | metab | | |
| PC.ae.C42.4 | lipid | Glycerophos- | phospholipid | 0.041074039 | −0.25932464 |
| | | phocholine | metab | | |
| TG.18.1_36.4. | lipid | triglyceride | fatty acid | 0.041074039 | 0.709557129 |
| | | | transport | | |
| Pyruvate | organic | α-keto acid | energy | 0.042963744 | −0.66791359 |
| | acid | | metab | | |
| Glu | amino | non-essential | amino acid | 0.042963744 | 0.583325922 |
| | acid | | metab | | |
| PC.aa.C30.0 | lipid | Glycerophos- | phospholipid | 0.042963744 | −0.50863753 |
| | | phocholine | metab | | |
| PC.ae.C34.1 | lipid | Glycerophos- | phospholipid | 0.042963744 | −0.25319553 |
| | | phocholine | metab | | |
| TG.16.0_38.4. | lipid | Triglyceride | fatty acid | 0.042963744 | 0.525614648 |
| | | | transport | | |
| TG.18.1 34.3. | lipid | Triglyceride | fatty acid | 0.042963744 | 0.673270577 |
| | | | transport | | |
| TG.18.2_36.3. | lipid | Triglyceride | fatty acid | 0.042963744 | 0.681074393 |
| | | | transport | | |
| TG.17.1 36.3. | lipid | Triglyceride | fatty acid | 0.044689913 | 0.615891674 |
| | | | transport | | |
| TG.17.1_36.4. | lipid | Triglyceride | fatty acid | 0.044689913 | 0.371947413 |
| | | | transport | | |
| Indoxylsulfate | organic | trp metab | amino acid | 0.048570682 | 0.677267378 |
| (Ind•SO₄) | sulfuric acid | | metab | | |
| DG.16.1_18.1. | lipid | Diacylglycerol | fatty acid | 0.048570682 | 0.528481227 |
| | | | transport | | |
| TG.20.0_32.4. | lipid | Triglyceride | fatty acid | 0.048570682 | 0.36786076 |
| | | | transport | | |

Note: parenthetical values indicate the length of fatty acid side chains. For FA the first number is the length of the side chain and the second number separated by a period is the degree of unsaturation in that side chain. Degree of unsaturation is the same as the number of double bonds. For PC, the number includes the total number of carbons in two fatty acid side chains. For DG the numbers indicate the chain lengths and degrees of unsaturation of the two fatty acid side chains. For TGs the first number indicates the chain length and degree of unsaturation of one chain and the second numbers indicate the sum of the chain lengths and degrees of unsaturation of the second and third fatty acid side chains.

“Mass spectrometry” (MS) is a technique for measuring and analyzing molecules that involves ionizing and/or fragmenting a target molecule, then analyzing the fragments, based on their mass/charge ratios, to produce a mass spectrum that serves as a “molecular fingerprint.” Several commonly used methods to determine the mass to charge ratio of an ion are known, some measuring the interaction of the ion trajectory with electromagnetic waves, others measuring the time an ion takes to travel a given distance, or a combination of both. The data from these fragment mass measurements can be searched against databases to obtain definitive identifications of target molecules.

“Monitoring” with reference to FA refers to a method or process of determining the severity or degree of FA or stratifying FA based on risk and/or probability of mortality. In some embodiments, monitoring relates to a method or process of determining the therapeutic efficacy of a treatment being administered to a subject.

“Neurological phenotype” refers to neurological features or disorders like loss of joint position and vibration sense in the limbs typical features of ataxia (which mostly results from degeneration of the dorsal root ganglia neurons, spinocerebellar tract, the cerebellar dentate nucleus), dysarthria (dentate nucleus involvement), vision with saccadic pursuit (optic nerve atrophy and dentate nucleus involvement) and hearing loss (central nerve involvement suggested by abnormal brainstem evoked auditory potentials), dysphagia (dentate nucleus involvement), areflexia (degeneration of peripheral nerves resulting of neuronal loss within DRG) and pyramidal syndrome and weakness identified with Babinski sign (dying back in cortico-spinal tracts).

“Nuclear magnetic resonance” (NMR) spectroscopy is a technique for determining the molecular structure and concentration of molecules. In the NMR analysis of metabolites, the biological samples are maintained in solution and then placed in a large magnetic field. The magnetic field is typically generated by a cryogenically cooled superconducting magnet, but lower magnetic fields including those generated by room temperature electromagnets or rare earth magnets can also be used. The samples are exposed to a band of radiofrequency (RF) waves which are absorbed by the molecules and excites the nuclei of the molecules. The typical focus of these experiments is on the excitation of hydrogen nuclei (also referred to as protons) and nuclei of carbon, in particular the C-13 isotope. After the RF excitation is turned off, a receiver coil then detects the RF energy that is released by the nuclei as they return from the excited state. Following Fourier transformation of these signals, an NMR spectrum is generated containing spectral peaks that, in different combinations, represent the sum of the metabolites in the sample. The resulting NMR spectrum can then be compared with reference spectra to both identify and quantify the different metabolites in the sample.

“Pediatric” refers to a subject under 18 years of age, while an adult subject is 18 or older.

“Physical or cognitive performance” refers to a subject's ability to perform certain physical or mental tasks. Physical performance includes, e.g., the ability to walk, run, balance, and coordinate various muscles, and spatial awareness. Cognitive performance includes, e.g., logic reasoning, touch sensitivity, and hearing and vision acumen.

“Predicting outcome” and “outcome risk stratification” with reference to FA refers to a method or process of prognosticating a patient's risk of a certain outcome. In some embodiments, predicting an outcome relates to monitoring the therapeutic efficacy of a treatment being administered to a subject. In some embodiments, predicting an outcome relates to determining a relative risk of mortality. Such mortality risk can be high risk, moderate risk, moderate-high risk, moderate-low risk, or low risk. Alternatively, such mortality risk can be described simply as high risk or low risk, corresponding to high risk of death or high likelihood of survival, respectively.

“Ratio” refers to a calculable relationship used to compare amounts of the concentration levels of two or more validated low molecular weight metabolic biomarkers. A ratio may be a direct proportion or inverse proportion (e.g., a first amount divided by a second amount or the second amount divided by the first amount, respectively). A ratio may be weighted and/or normalized (either the numerator, the denominator, or both). The two amounts may be physical quantities or arbitrary values that correspond to physical quantities. For example, a ratio may be calculated from two concentration levels (i.e., in arbitrary units) in two biomarkers (e.g., validated low molecular weight metabolic biomarkers) measured by a mass spectrometry technique.

“Reference concentration level” refers to the amount of a biomarker established for a subject with no FA, the amount of biomarker established for an asymptomatic subject, the amount of biomarker established for an early-symptomatic subject, a symptomatic subject or a late-symptomatic subject as determined by one skilled in the art using established methods as described herein, and/or a known level of a biomarker obtained from literature. The reference concentration level of the biomarker can further refer to the level of biomarker for a population of subjects. The reference concentration level of the biomarker can also refer to the concentration level of the biomarker established for a particular stage in progression of FA, for a subject at a particular stage of treatment for FA (e.g., start and finish of treatment), for a particular phenotype associated with FA, for a stage of progression of a particular phenotype associated with FA, or a particular outcome associated with FA. The reference concentration level of the biomarker can also refer to the concentration level of the biomarker obtained from the subject to which the method is applied. As such, the change within a subject from visit to visit can indicate a change the severity or degree of FA or a stratification in risk associated with FA.

“Safe and effective amount” means an amount sufficient to treat a FA or an associated phenotype or a condition but low enough to avoid serious side effects (at a reasonable benefit/risk ratio) within the scope of sound medical judgment. A safe and effective amount will vary with the particular compound chosen (e.g., consider the potency, efficacy, and half-life of the compound); the route of administration chosen; the condition being treated; the severity of the condition being treated; the age, size, weight, and physical condition of the subject being treated; the medical history of the subject to be treated; the duration of the treatment; the nature of concurrent therapy; the desired therapeutic effect; and like factors. In some embodiments, the present disclosure may be used to determine a safe and effective amount of a test compound.

“Score” denotes a value, in particular a quantitative value, generated from metabolic biomarker data by means of any mathematical transformation or by subjecting to any mathematical equation and comparing these data to data or mathematically transformed or processed data of a reference or control population.

“Skeletal or cardiac abnormalities” refers to the abnormalities in a subject's skeletal system or heart that are caused by FA. Skeletal abnormalities associated with FA include, but are not limited to, inversion of the feet, a shortened foot with a high arch, and scoliosis. Cardiac abnormalities associated with FA include, but are not limited to, hypertrophic cardiomyopathy, an enlargement of cardiac muscles, and arrhythmia.

“Stabilization of a phenotype associated with Friedreich ataxia” refers to slowing or halting development of the phenotype or restoration of a normal phenotype.

“Subject” refers to a human subject as well as a non-human subject such as a non-human mammal. Thus, various veterinary applications are contemplated in which case the subject may be a non-human mammal (e.g., a feline, a porcine, an equine, a bovine, and the like). The concepts described are also applicable to plants. “Subject” does not denote a particular age or sex and, thus, includes adult, pediatric and newborn subjects, whether male or female. “Normal control subjects” or “normal controls” means healthy subjects who are clinically free of FA. “Normal control sample” or “control sample” refers to a biological sample that has been obtained from a normal control subject.

“Target tissue” refers to any tissue that is affected by FA. In some embodiments, a target tissue includes those tissues that display disease-associated pathology, symptom, or feature. Examples of target tissues include, but are not limited to, heart tissue, spinal cord tissue, liver tissue, pancreatic tissues, and skeletal muscle.

“Test compound” can be any chemical compound, for example, a macromolecule (e.g., a polypeptide, a protein complex, glycoprotein, or a nucleic acid) or a small molecule (e.g., an amino acid, a nucleotide, an organic or inorganic compound). A test compound can have a formula weight of less than about 10,000 grams per mole, less than 5,000 grams per mole, less than 1,000 grams per mole, or less than about 500 grams per mole. The test compound can be naturally occurring (e.g., an herb or a natural product), synthetic, or can include both natural and synthetic components. Examples of test compounds include peptides, peptidomimetics (e.g., peptoids), amino acids, amino acid analogs, polynucleotides, polynucleotide analogs, nucleotides, nucleotide analogs, and organic or inorganic compounds, e.g., hetero-organic or organometallic compounds. A test compound may be a drug or therapeutic agent currently in development and/or in clinical trials.

“Treat,” “treating” or “treatment” refer to an action to obtain a beneficial or desired clinical result including, but not limited to, alleviation or amelioration of one or more signs or symptoms of FA (e.g., regression, partial or complete), diminishing the extent of disease, stability (i.e., not worsening, achieving stable disease) of the state of disease, amelioration or palliation of the disease state, diminishing rate of or time to progression, and remission (whether partial or total). “Treatment” of FA can also mean prolonging survival as compared to expected survival in the absence of treatment. Treatment need not be curative. In certain embodiments, treatment includes one or more of a decrease in pain or an increase in the quality of life (QOL) as judged by a qualified individual, e.g., a treating physician, e.g., using accepted assessment tools of pain and QOL. In certain embodiments, a decrease in pain or an increase in the QOL as judged by a qualified individual, e.g., a treating physician, e.g., using accepted assessment tools of pain and QOL is not considered to be a “treatment” of the disease. “Treat” covers any treatment in a mammal and includes: (a) preventing FA from occurring in a subject which may be predisposed to the disease but has not yet been diagnosed as having it; (b) inhibiting FA, i.e., arresting its development; or (c) relieving FA, i.e., causing regression of the disease. The therapeutic treatment may be administered before, during or after the onset of the disease. The inventive therapy may be administered during a symptomatic stage of the disease, and in some cases after the symptomatic stage of the disease.

“Validated” refers to a statistically significant difference in a level of a metabolite biomarker in a biological sample from a subject and a reference or control level of at least about 5%, or greater, e.g., at least about 10%, 15%, 20%, 25%. When performing multivariate statistical analysis during biomarker discovery in metabolomics, corrected p-values are often used to correct for multiple hypothesis testing to reduce false discoveries, such as the use of a false discovery rate (q<0.05) or a more conservative Bonferroni correction. The determination of statistical significance is well-established in the art. Statistical significance is attained when a p-value is less than the significance level. The p-value is the probability of observing an effect given that the null hypothesis is true whereas the significance or alpha level is the probability of rejecting the null hypothesis given that it is true.

Once a biological sample is obtained, it is analyzed to determine the concentration level of the selected validated metabolite biomarker(s) in the sample. Before analysis, the sample may be subject to processing such as extraction, filtration, centrifugation or other sample preparation techniques to provide a sample that is suitable for further analysis. For example, biological fluids may be filtered or centrifuged (e.g., ultracentrifugation) to remove solids from the sample to facilitate analysis. As one of skill in the art will appreciate, biomarker level may be determined using one of several techniques established in the art that would be suitable for detecting such biomarkers, e.g., polar metabolites, in a biological sample, including mass spectrometry, chromatographic techniques such as high performance liquid chromatography and gas chromatography, immunoassay or enzyme-based assays with colorimetric, fluorescence or radiometric detection. As one of skill in the art will appreciate, metabolite biomarkers may be analyzed directly or may be chemically derivatized for analysis and may be analyzed by comparison against stable-isotope internal standards.

In some embodiments, metabolite biomarker detection is accomplished using a mass spectrometry (MS)-based method. Suitable MS-based methods include direct infusion-mass spectrometry, electrospray ionization (ESI)-MS, desorption electrospray ionization (DESI)-MS, direct analysis in real-time (DART)-MS, atmospheric pressure chemical ionization (APCI)-MS, electron impact (EI) or chemical ionization (CI), as well as MS-based methods coupled with a separation technique, such as liquid chromatography (LC-MS), gas chromatography (GC-MS), or capillary electrophoresis (CE-MS) mass spectrometry. In some embodiments, metabolite biomarker detection is accomplished using a combination of NMR and LC-MS.

In some embodiments, there may be chemical derivatization of certain metabolites to improve the detection, thus they would no longer be endogenous metabolites. The metabolites may then be measured using a specific protocol on an analytical instrument, such as a mass spectrometer (LC-MS or GC-MS) or NMR. It is also possible that the measurement could be carried out by, for example, a custom set of chemical assays.

In some embodiments, a computer-based analysis program is used to translate the raw data generated by the assay (e.g., the presence, absence, or amount of a FA specific metabolite) into data of predictive value for a clinician. The clinician can access the predictive data using any suitable means. Thus, in some embodiments, the present disclosure provides the further benefit that the clinician, who is not likely to be trained in metabolite analysis, need not understand the raw data. The data is presented directly to the clinician in its most useful form. The clinician is then able to immediately utilize the information to optimize the care of the subject. The present disclosure contemplates any method capable of receiving, processing, and transmitting the information to and from laboratories conducting the assays, information provides, medical personal, and subjects. For example, in some embodiments of the present disclosure, a sample (e.g., a biopsy or a blood, urine or plasma sample) is obtained from a subject and submitted to a profiling service (e.g., clinical lab at a medical facility, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a urine sample) and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication systems). Once received by the profiling service, the sample is processed and a profile is produced (e.g., metabolic profile), specific for the diagnostic or prognostic information desired for the subject.

In embodiments, the method performance does not simply rely on the presence or direct concentrations of specific metabolites, but rather the diagnostic panel is derived from the application of machine learning methods such as Random Forest and Support Vector Machines to provide a composite measure that relates to disease severity and/or response to pharmaceutical interventions. As the panel includes metabolites from different biological pathways, it is possible that this diagnostic could also be used to subtype the disease progression or drug response. For example, some components of the panel could suggest that derangements in 1-carbon metabolism have been normalized, but the fatty acid signals are still perturbed. This could inform the application of personalized therapeutic strategies.

The profile data is then prepared in a format suitable for interpretation by a treating clinician. For example, rather than providing raw data, the prepared format may represent a diagnosis or risk assessment for the subject, along with recommendations for particular treatment options. The profile data may also be used by the treating clinician to measure response to a therapy intended to treat, for example, a neurological or cardiac phenotype associated with FA. The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or subject. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers. In some embodiments, the subject is able to directly access the data using the electronic communication system. The subject may choose further intervention or counseling based on the results. In some embodiments, the data is used for research use. For example, the data may be used to further optimize the inclusion or elimination of markers as useful indicators of a particular condition or stage of disease.

In some embodiments, methods and systems of the invention, can be embodied as a computer implemented process or processes for performing such computer-implemented process or processes, and can also be embodied in the form of a tangible storage medium (i.e., non-transitory computer readable medium) containing a computer program or other machine-readable instructions (herein “computer program”), where when the computer program is loaded into a computer or other processor (herein “computer”) and/or is executed by the computer, the computer becomes an apparatus for practicing the process or processes. Storage media for containing such computer program include, for example, floppy disks and diskettes, compact disk (CD)-ROMs (whether or not writeable), DVD digital disks, RAM and ROM memories, computer hard drives and back-up drives, external hard drives, solid state drives, “thumb” drives, and any other storage medium readable by a computer. The process or processes can also be embodied in the form of a computer program, for example, whether stored in a storage medium or transmitted over a transmission medium such as electrical conductors, fiber optics or other light conductors, or by electromagnetic radiation, where when the computer program is loaded into a computer and/or is executed by the computer, the computer becomes an apparatus for practicing the process or processes. The process or processes may be implemented on a general-purpose microprocessor or on a digital processor specifically configured to practice the process or processes. When a general-purpose microprocessor is employed, the computer program code configures the circuitry of the microprocessor to create specific logic circuit arrangements. Storage medium readable by a computer includes medium being readable by a computer per se or by another machine that reads the computer instructions for providing those instructions to a computer for controlling its operation.

Aspects of the present disclosure can be described as embodiments in any of the following enumerated clauses. It will be understood that any of the described embodiments can be used in connection with any other described embodiments to the extent that the embodiments do not contradict one another.

1. A method to assess a subject having FA, the method comprising: (i) determining a concentration level of a panel of at least two validated low molecular weight metabolic biomarkers in a biological sample from the subject; (ii) identifying a difference between the determined concentration level of the at least two metabolic biomarkers and a reference concentration level of the at least two metabolic biomarkers; and, (iii) assessing the subject based on the identified difference.

2. A method according to the preceding clause, where one or more elements of the method are computer-implemented.

3. A method according to any of the preceding clauses, further comprising assigning a score to the subject that represents the assessment.

4. A method according to any of the preceding clauses, where the subject is human.

5. A method according to any of the preceding clauses, where the biological sample is blood.

6. A method according to any of the preceding clauses, where the biological sample is blood plasma or blood serum.

7. A method according to any of the preceding clauses, where the biological sample is tissue.

8. A method according to any of the preceding clauses, where the biological sample is target tissue.

9. A method according to any of the preceding clauses, where the panel of at least two validated low molecular weight metabolic biomarkers is a panel of from five to ten validated low molecular weight metabolic biomarkers.

10. A method according to any of the preceding clauses, where the panel of at least two validated low molecular weight metabolic biomarkers is a panel of more than ten validated low molecular weight metabolic biomarkers.

11. A method according to any of the preceding clauses, where the panel of at least two low molecular weight metabolic biomarker are selected from a biomarker in Table 1.

12. A method according to any of the preceding clauses, where the panel of at least two metabolic biomarkers are metabolic biomarkers associated with at least two different metabolic pathways.

13. A method according to any of the preceding clauses, where the panel of at least two metabolic biomarkers are metabolic biomarkers associated with the same metabolic pathway.

14. A method according to any of the preceding clauses, where the panel of at least two metabolic biomarkers are selected from the group consisting of an energy metabolism biomarker, a fatty acid metabolism biomarker, an amino acid metabolism biomarker, a urea cycle biomarker, a 1-carbon metabolism biomarker, a cholesterol metabolism biomarker, a phospholipid metabolism biomarker, a fatty acid storage/transport biomarker and a lipoprotein metabolism biomarker.

15. A method according to any of the preceding clauses, where the panel further comprises at least one metabolic biomarker that is not an energy metabolism biomarker, a fatty acid metabolism biomarker, an amino acid metabolism biomarker, a urea cycle biomarker, a 1-carbon metabolism biomarker, a cholesterol metabolism biomarker, a phospholipid metabolism biomarker, a fatty acid storage/transport biomarker or a lipoprotein metabolism biomarker.

16. A method according to any of the preceding clauses, where the metabolic biomarker is an energy metabolism biomarker.

17. A method according to any of the preceding clauses, where the metabolic biomarker is an energy metabolism biomarker selected from the group consisting of acetate, glucose, lactate and pyruvate.

18. A method according to any of the preceding clauses, where the metabolic biomarker is a fatty acid metabolism biomarker.

19. A method according to any of the preceding clauses, where the metabolic biomarker is a fatty acid metabolism biomarker selected from the group consisting of carnitine, malonylcarnitine, hydroxybutyrylcarnitine, hydroxypropionylcarnitine, hexenoylcarnitine, FA 20.3 and dodecanedioic acid.

20. A method according to any of the preceding clauses, where the metabolic biomarker is an amino acid metabolism biomarker.

21. A method according to any of the preceding clauses, where the metabolic biomarker is a canonical amino acid or a non-canonical amino acid.

22. A method according to any of the preceding clauses, where the metabolic biomarker is an amino acid metabolism biomarker selected from the group consisting of glutamate, leucine, lysine, threonine and indoxylsulfate.

23. A method according to any of the preceding clauses, where the metabolic biomarker is a urea cycle biomarker.

24. A method according to any of the preceding clauses, where the metabolic biomarker is a urea cycle biomarker selected from the group consisting of arginine, citrulline and ornithine.

25. A method according to any of the preceding clauses, where the metabolic biomarker is a 1-carbon metabolism biomarker.

26. A method according to any of the preceding clauses, where the metabolic biomarker is a 1-carbon metabolism biomarker selected from the group consisting of formate, homocysteine, hypoxanthine, choline, sarcosine and histamine.

27. A method according to any of the preceding clauses, where the metabolic biomarker is a cholesterol metabolism biomarker.

28. A method according to any of the preceding clauses, where the metabolic biomarker is a cholesterol metabolism biomarker selected from the group consisting of cholic acid and cholesterol ester 20:0.

29. A method according to any of the preceding clauses, where the metabolic biomarker is a phospholipid metabolism biomarker.

30. A method according to any of the preceding clauses, where the metabolic biomarker is a phospholipid metabolism biomarker selected from the group consisting of PC.aaC26.0, PC.aaC30.0, PC.aaC32.0, PC.aaC34.2, PC.aaC36.2, PC.aaC40.1, PC.aaC40.2, PC.aeC34.1, PC.aeC34.2, PC.aeC34.3, PC.aeC36.2, PC.aeC36.3, PC.aeC36.4, PC.ae42.4, PC.aeC44.4 and SM.C24.0.

31. A method according to any of the preceding clauses, where the metabolic biomarker is a fatty acid storage/transport biomarker.

32. A method according to any of the preceding clauses, where the metabolic biomarker is a fatty acid storage/transport biomarker selected from the group consisting of Cer.d18.0.22.0, DG.16.1_18.1, DG.16.1_18.2, TG.16.0_28.2, TG.16.0_38.4, TG.20.0_32.4, TG.17.1_36.3, TG.17.1_36.4, TG.18.1_34.3, TG.18.1_36.4, TG.18.1_36.5, TG.18.2_36.3, TG.18.3_34.2, TG.18.3_36.2, TG.20.0_32.3, and TG.20.0_32.4.

33. A method according to any of the preceding clauses, where the metabolic biomarker is a lipoprotein metabolism biomarker.

34. A method according to any of the preceding clauses, where the metabolic biomarker is a lipoprotein metabolism biomarker selected from the group consisting of LLP3, LSP3, HDLP3, HLP3, LZ3 HZ3 and ELP_HDLC.

35. A method according to any of the preceding clauses, where the identified difference in biomarker concentration is independently selected for each biomarker from an increased concentration and a decreased concentration.

36. A method according to any of the preceding clauses, where the reference concentration level is determined from a healthy subject.

37. A method according to any of the preceding clauses, further comprising calculating a ratio of the concentration levels of the at least two validated low molecular weight metabolic biomarkers.

38. A method according to any of the preceding clauses, where the concentration level of the at least two low molecular weight metabolic biomarkers in the biological sample is determined by mass spectrometry.

39. A method according to any of the preceding clauses, where the concentration level of the at least two metabolic biomarkers in the biological sample is determined by a chromatography and/or a spectrometry method.

40. A method according to any of the preceding clauses, where the concentration level of the at least two low molecular weight metabolic biomarkers is determined by chromatography comprising GC, LC, HPLC, and UPLC; spectroscopy comprising UV/Vis, IR, and NMR; and mass spectrometry comprising ESI-QqQ, ESI-QqTOF, MALDI-QqQ, MALDI-QqTOF, and MALDI-TOF-TOF.

41. A method according to any of the preceding clauses, where the assessing step includes determining a stage of progression of FA in the subject.

42. A method according to any of the preceding clauses, where the assessing step includes differentially and/or comparatively diagnosing between symptomatic FA and late-symptomatic FA.

43. A method according to any of the preceding clauses, where the subject is undergoing a treatment for FA and the assessing step includes determining whether to continue treatment, to discontinue treatment, or to alter treatment based on presence, absence, or the concentration level of the at least two metabolic biomarkers in the panel.

44. A method according to any of the preceding clauses, where the subject is undergoing a treatment for FA and the assessing step includes monitoring an effect of the treatment on the subject.

45. A method according to any of the preceding clauses, further comprising comparing the identified differences in a biological sample taken from a subject at two or more points in time.

46. A method according to any of the preceding clauses, further comprising comparing the identified differences in a biological sample taken from a subject at two or more points in time, where a change in the identified differences toward a phenotype profile, is interpreted as a progression toward the phenotype.

47. A method according to any of the preceding clauses, further comprising comparing the identified differences in a biological sample taken from a subject before and after a treatment or during a course of treatment.

48. A method according to any of the preceding clauses, further comprising comparing the identified differences in a biological sample taken from a subject before and after a treatment or during a course of treatment, where a change in biomarker profile over time toward an asymptomatic profile or to a stable profile is interpreted as efficacy.

49. A method according to any of the preceding clauses, further comprising: (i) providing a recommended treatment; and, (ii) administering the treatment to the subject.

50. A method according to any of the preceding clauses, where the assessing includes monitoring FA or a phenotype associated with FA of the subject.

51. A method according to any of the preceding clauses, where the assessing includes assessing a phenotype associated with FA of the subject.

52. A method according to any of the preceding clauses, where the assessing includes assessing a phenotype associated with FA of the subject and the phenotype is physical or cognitive performance.

53. A method according to any of the preceding clauses, where the assessing includes assessing a phenotype associated with FA of the subject and the phenotype is skeletal or cardiac abnormalities.

54. A method according to any of the preceding clauses, where the assessing includes assessing stabilization of a phenotype associated with FA.

55. A method according to any of the preceding clauses, where the assessing includes assessing stabilization of a cardiac phenotype associated with FA.

56. A method according to any of the preceding clauses, where the assessing includes assessing stabilization of a neurological phenotype associated with FA.

57. A method according to the any of the preceding clauses, where the assessing includes determining a stage in progression of a phenotype.

58. A method according to the any of the preceding clauses, where the assessing includes determining a stage in progression of an ataxia phenotype.

59. A method according to the any of the preceding clauses, where the assessing includes determining a stage in progression of a cardiac phenotype.

60. A method according to any of the preceding clauses, where the assessing includes determining a stage in progression of a cardiac phenotype and cardiac phenotype is heart failure.

61. A method according to any of the preceding clauses, where the assessing includes determining a stage in progression of a cardiac phenotype, the cardiac phenotype is heart failure, and the determined stage is congenital heart failure.

62. A method according to any of the preceding clauses, where the assessing includes predicting whether a phenotype will change.

63. A method according to any of the preceding clauses, where the assessing includes predicting whether a phenotype will improve.

64. A method according to any of the preceding clauses, where the assessing includes predicting whether a phenotype will worsen.

65. A method according to any of the preceding clauses, where the assessing is assessing a phenotype associated with FA selected from the group consisting of an ataxia phenotype, a muscle weakness phenotype, a loss of coordination phenotype, a vision impairment phenotype, a slurred speech phenotype, a spine curvature phenotype, a diabetes phenotype, a cardiac phenotype, a central neurological phenotype, and a peripheral neurological phenotype.

66. A method according to any of the preceding clauses, where the assessing is assessing a cardiac phenotype associated of the subject.

67. A method according to any of the preceding clauses, where the assessing includes predicting an outcome for the subject.

68. A method according to any of the preceding clauses, where the assessing includes predicting an outcome for the subject and the outcome is survival.

69. A method according to any of the preceding clauses, where the assessing includes predicting an outcome for the subject and the outcome is survival from heart disease.

70. A method according to any of the preceding clauses, where the assessing includes determining a treatment course of action.

71. A method according to any of the preceding clauses, where the assessing includes predicting a treatment outcome for the subject.

72. A method according to any of the preceding clauses, where the assessing includes predicting treatment outcome for the subject and the treatment prediction outcome is selected from the group consisting of complete response to treatment, partial response to treatment and non-response to treatment.

73. A method according to any of the preceding clauses, where the assessing includes predicting treatment outcome for a pharmaceutical intervention.

74. A method according to any of the preceding clauses, where the assessing includes predicting a likelihood that the subject will respond to a treatment.

75. A method according to any of the preceding clauses, where the assessing includes using a model generated by a machine learning algorithm to assess the subject based on an identified difference between the determined concentration level of the at least two metabolic biomarkers and a reference concentration level of the at least two metabolic biomarkers in the panel.

76. A method according to any of the preceding clauses, where the assessing includes using an analytical characteristic associated with the subject.

77. A method according to any of the preceding clauses, where the assessing includes using a plurality of analytical characteristics associated with the subject.

78. A method according to any of the preceding clauses, where the assessing includes using an analytical characteristic associated with the subject that is a clinical characteristic.

79. A method according to any of the preceding clauses, where the assessing includes using an analytical characteristic associated with the subject that is a clinical characteristic selected from the group consisting of age, age at diagnosis, and gender.

80. A method according to any of the preceding clauses, where the assessing includes using an analytical characteristic associated with the subject that is a molecular characteristic.

81. A method according to any of the preceding clauses, where the assessing includes using an analytical characteristic associated with the subject that is a molecular characteristic that is not a metabolic biomarker.

82. A method according to any of the preceding clauses, where the assessing includes using an analytical characteristic associated with the subject that is a genetic marker molecular characteristic.

83. A method to assess efficacy of a test compound to treat FA, the method comprising: (i) determining a concentration level of a panel of at least two validated low molecular weight metabolic biomarkers in a cell contacted with the test compound; (ii) identifying a difference between the determined concentration level of the at least two metabolic biomarkers and a reference concentration level of the at least two metabolic biomarkers; and, (iii) assessing the efficacy of the test compound based on the identified difference.

84. A method according to the preceding clause, where one or more elements of the method are computer-implemented.

85. A method according to any of the preceding clauses, where the contacted cell is in a target tissue.

86. A method according to any of the preceding clauses, where the assessing is a high-throughput parallel screen of a test compound library.

87. A method according to any of the preceding clauses, where the contacted cell is isolated from a multicellular organism model of FA.

88. A method according to any of the preceding clauses, where the panel of at least two low molecular weight metabolic biomarker are selected from a biomarker in Table 1.

89. A method to assess efficacy of a test compound to treat FA, the method comprising: (i) determining a concentration level of a panel of at least two validated low molecular weight metabolic biomarkers in a biological sample from a subject administered the test compound; (ii) identifying a difference between the determined concentration level of the at least two metabolic biomarkers and a reference concentration level of the at least two metabolic biomarkers; and, (iii) assessing the efficacy of the test compound based on the identified difference.

90. A method according to the preceding clause, where one or more elements of the method are computer-implemented.

91. A method according to any of the preceding clauses, where the subject is an animal.

92. A method according to any of the preceding clauses, where the subject is a multicellular organism.

93. A method according to any of the preceding clauses, where the panel of at least two low molecular weight metabolic biomarker are selected from a biomarker in Table 1.

94. A diagnostic kit for assessing a subject for an attribute of FA, where the attribute of FA is associated with a low molecular weight metabolic biomarker profile, the kit comprising: a container comprising a panel of at least two validated low molecular weight metabolic biomarker internal standards having a purity greater than 98.0%, where the container is configured to receive a biological sample from the subject and to be sealed with a sealing member after receiving the biological sample.

95. A diagnostic kit according to the preceding clause, the kit further comprising instructions.

96. A diagnostic kit according to any of the preceding clauses, where the attribute is a positive or negative diagnostic of FA in the subject.

97. A diagnostic kit according to any of the preceding clauses, where the panel of at least two low molecular weight metabolic biomarker are selected from a biomarker in Table 1.

98. A method to identify a candidate low molecular weight metabolic biomarker panel that differentially and/or comparatively diagnoses a subject having FA, the method comprising: (i) obtaining a first biological sample from a first subject having a first phenotype associated with FA and a second biological sample from a second subject having a second phenotype associated with FA; (ii) determining a concentration level of at least two low molecular weight metabolic biomarkers in the first biological sample, the second biological sample, and a reference concentration level of the at least two metabolic biomarkers; and, (iii) identifying two or more validated low molecular weight metabolic biomarkers that are present in a statistically significant, different level in the first and second biological samples, where the different level is characterized by an area under the receiver operator characteristic (ROC) curve (AUC) ranging from 0.60 to 1.00.

99. A method according to the preceding clause, where one or more elements of the method are computer-implemented.

100. A method according to any of the preceding clauses, where the panel of at least two low molecular weight metabolic biomarker are selected from a biomarker in Table 1.

101. A method to diagnose FA in a subject, the method comprising: (1) determining a concentration level of a panel of at least two validated low molecular weight metabolic biomarkers in a biological sample from the subject; (ii) identifying a difference between the determined concentration level of the at least two metabolic biomarkers and a reference concentration level of the at least two metabolic biomarkers; and, (iii) diagnosing FA in the subject based on the identified difference.

102. A method to evaluate effectiveness of a FA treatment in a subject that has been diagnosed with FA, the method comprising: (1) determining a concentration level of a panel of at least two validated low molecular weight metabolic biomarkers in a biological sample from the subject; (ii) identifying a difference between the determined concentration level of the at least two metabolic biomarkers and a reference concentration level of the at least two metabolic biomarkers; and, (iii) assessing effectiveness of the FA treatment based on the identified difference.

103. A method according to the preceding clause, where one or more elements of the method are computer-implemented.

104. A method according to any of the preceding clauses, where the panel of at least two low molecular weight metabolic biomarker are selected from a biomarker in Table 1.

105. An electronic system to assess a subject having FA, the system comprising: (i) a memory; and, (ii) a processor in communication with the memory where the processor is operable to execute instructions for obtaining a concentration level of a panel of at least two validated low molecular weight metabolic biomarkers in a biological sample from the subject; identifying a difference between the determined concentration level of the at least two metabolic biomarkers and a reference concentration level of the at least two metabolic biomarkers; and, assessing the subject based on the identified difference.

106. A system according to the preceding clause, further comprising a display device and where the assessment is displayed on the display device.

107. A system according to any of the preceding clauses, where the panel of at least two low molecular weight metabolic biomarker are selected from a biomarker in Table 1.

108. A system to assess a subject having FA; the system including a processor operable to execute one or more computer programs, the one or more computer programs comprising instructions for carrying out a method to assess a subject having FA, the method comprising: (i) identifying a difference between a determined concentration level of a panel of at least two metabolic biomarkers in a biological sample from the subject and a reference concentration level of the at least two metabolic biomarkers; and, (ii) assessing the subject based on the identified difference.

109. A non-transitory computer readable storage medium according to the preceding clause, where the panel of at least two low molecular weight metabolic biomarker are selected from a biomarker in Table 1.

### EXAMPLES

Examples related to the present disclosure are described below. In most cases, alternative techniques can be used. The examples are intended to be illustrative and are not limiting or restrictive of the scope of the invention as set forth in the claims.

### Introduction

As discussed above, FA is fundamentally a metabolic disease with systemic impact and is especially disruptive for the heart and nervous system. The protein frataxin (FXN) is predicted to have a role in the biosynthesis of ubiquitous iron-sulfur clusters and reduced FXN has the potential to profoundly alter cellular metabolism. The gene defect underlying FA was identified in 1996 as a large GAA triplet expansion, frequently>800 repeats, in the first intron of the human FXN gene (FRDA) on chromosome 9q21.11. A correlation between GAA repeat number and the onset and severity of clinical symptoms has been reported, with higher repeat numbers being associated with earlier onset and more rapid rate of disease progression. The exact function of frataxin is not fully established but it is known to play a significant role in iron metabolism, iron storage, and iron-sulfur (Fe/S) cluster biogenesis with numerous downstream effects including alterations to cellular energy metabolism. The Fe/S proteins are ubiquitous components that have a diverse set of functions and are found in virtually all living cells. A number of enzymes that are critical to cellular energy metabolism are dependent on Fe/S clusters including mitochondrial electron transport chain complexes I, II, and III, aconitase in the Krebs Cycle, and electron transport flavoprotein (ETF). The resulting defect in the electron transport chain and Krebs Cycle also causes a disruption in the NAD/NADH ratio, which is associated with heavy acetylation of multiple proteins throughout the mitochondrial matrix. This may be associated with impaired oxidation of fatty acids and a shift to glycolysis in energy dependent tissues, such as heart. In its absence or with reduced levels, ATP production within mitochondria is severely decreased with multiple metabolic effects and significant alterations in nuclear gene expression.

Multiple therapeutic approaches for the treatment of FA are in development, but a key limitation is the lack of a way to assess the activity of FXN, and thus an inability to monitor a biochemical response to an intervention, in a timely fashion. In the absence of an identified function for FXN, it is difficult to measure the pharmacodynamic response to a therapeutic drug to establish a dose-response relationship. Thus, trials have used clinical endpoints, such as the modified FA Rating Scale (mFARS), but power analyses and recent assessment of progression characteristics have shown that trials using these endpoints need to be conducted for at least 1 to 2 years. What is needed to accelerate drug discovery are a set of biomarkers that represent the deranged pathophysiology in FA and can rapidly and quantitatively detect the efficacy of an intervention.

In this study, metabolomics techniques were applied to human FA patients. The field of metabolomics involves the comprehensive analysis of small molecules reflecting the substrates, intermediates, and products of cellular metabolism. Given the severe metabolic perturbations predicted and observed in FA, a comprehensive, multi-platform metabolomics approach was used to identify a highly distinctive metabolic signature in FA patients including specific alterations to one-carbon (1C) metabolism related pathways. This multiplatform, mass spectrometry and NMR-based approach provided a unique and comprehensive picture of the serum metabolome including a wide range of lipids as well as hydrophilic metabolites. Overall, this study identified an intriguing association of many of the most significantly altered metabolites with pathways involved in one-carbon metabolism including the folate cycle, methionine salvage, and purine nucleotide salvage and synthesis. Importantly, this metabolite panel accurately distinguishes controls from FA patients with high sensitivity and specificity, showing its usefulness to serve as a biomarker panel to evaluate disease progression and efficacy of therapeutic interventions. Receiver operator characteristics analysis of the top 10 biomarkers yielded an area under the curve of 0.96 (CI=0.75-1.0).

This study demonstrated that dysregulated metabolism caused by FA presents a unique metabolite profile in blood of FA patients versus Controls (Con). Plasma from 10 FA and 10 age and sex matched Con subjects was analyzed by targeted mass spectrometry and untargeted NMR. This combined approach yielded quantitative measurements for 540 metabolites and identified 59 unique metabolites (55 from MS and 4 from NMR) that were significantly different between cohorts. Correlation-based network analysis revealed several clusters of pathway related metabolites including a cluster associated with one-carbon (1C) metabolism composed of formate, sarcosine, hypoxanthine, and homocysteine. Receiver operator characteristics analyses demonstrated an excellent ability to discriminate between Con and FA with AUC values>0.95. These results demonstrate the effectiveness of metabolomic analyses of human patients with FA. The metabolic perturbations, especially those related to 1C metabolism, enable the use of a valuable biomarker panel to quickly determine disease progression and response to therapy. The identification of dysregulated 1C metabolism also informs the search for new therapeutic targets related to this pathway.

### Materials and Methods

Study cohort. The study cohort was composed of 11 healthy controls (Con) and 10 patients with FA. Patient characteristics for all subjects in the study are shown in Table 2, infra. Of the FA subjects, 70% were on one or more cardiac medications including β-blockers, calcium channel blockers, or diuretics. Two FA subjects were also on medications to control angina. No FA or Con subjects were on medication for diabetes. Two FA subjects were on chronic medications for muscle spasm or pain, and 60% of FA subjects were on a medication intended to help slow the advance or treat the neurologic symptom of FA including Idebenone and one subject who was also on selegiline. The Con subjects were on none of these medications. The FA cohort was composed of 9 Caucasian and 1 Black subjects, whereas the Con cohort was composed of 10 Caucasian subjects. For the FA cohort, the average GAA repeat length on the shorter allele ranged from 158 repeats to >800, with an average of 612 repeats. The age at FA diagnosis ranged from 5 years (GAA repeat 400) to 23 years (GAA repeat=549), with an average age at diagnosis of 14.1±5.9 yr. There were no significant differences in age, sex, weight, height, or BMI between the groups.

TABLE 2

| | Control | Friedreich's |
|---|---|---|
| Parameter | (n = 11) | Ataxia (n = 10) |
| Age (years) | 28.0 ± 8.7 | 23.3 ± 5.4 |
| Biological Sex | 6F/5M | 5F/5M |
| Weight (kg) | 74.2 ± 10.8 | 75.7 ± 21.2 |
| Height (cm) | 170.4 ± 11.1 | 187.8 ± 8.3 |
| Body Mass Index (kg/m²) | 25.8 ± 6.3 | 25.3 ± 5.5 |

Baseline Medications

| | | |
|---|---|---|
| Cardiac | 0% | 70% |
| Pain Management | 0% | 20% |
| FA-specific | 0% | 60% |

Samples. Blood samples for metabolomics analysis were drawn into EDTA tubes, spun, and plasma was aliquoted and stored frozen (−80° C.) until use. Metabolic profiling of serum from all subjects in this study was carried out using a comprehensive, multi-platform approach that included a targeted mass spectrometry (MS) platform and an untargeted nuclear magnetic resonance (NMR)-based platform.

NMR-based metabolomics. Samples for NMR analyses were prepared using established protocols known to those skilled in the art. NMR data were acquired on a Bruker AVANCE III, 700 MHz NMR equipped with a cryogenically-cooled probe. Spectra were collected with a 1D NOESY pulse sequence covering 12 ppm. The spectra were digitized with 32768 points during a 3.9 s acquisition time. The mixing time was set to 100 ms, and the relaxation delay between scans was set to 2.0 s. The untargeted NMR analyses quantified 26 metabolites. The data was processed and analyzed using the Chenomx NMR Processor and Profilers software packages (Chenomx Inc., Edmonton, Alberta, Canada).

Targeted MS-based metabolomics. The targeted MS experiments used the Biocrates Q500 kit (Biocrates AG. Innsbruck, Austria) run on an AB Sciex 5500 QTRAP with an Agilent 1290 UPLC. Preparation of serum samples followed vendor protocols. This assay yielded quantitative measures of 495 metabolites. Data processing was carried out using the Biocrates MetIDQ software. Metabolites with concentrations of zero in more than 50% of the samples were excluded.

Data integration and biomarker analysis. Integration, statistical analysis and visualization of the metabolomics data was carried out using the VIIME platform along with custom scripts written in the R programming language. The network correlation analyses and correlogram were generate using the corrplot package in R. The network community analysis utilized the visualization capabilities in the igraph package. The receiver operator characteristic (ROC) analysis was carried out in MetaboAnalyst (version 5.0) using the multivariate exploratory analysis functions. ROC curves were generated by Monte-Carlo cross-validation with classification and ranking carried out using the Random Forests algorithm including two latent variables. Support Vector machine and PLS-DA exhibited similar results, as did other statistical and machine learning methods known to those skilled in the art.

### Results

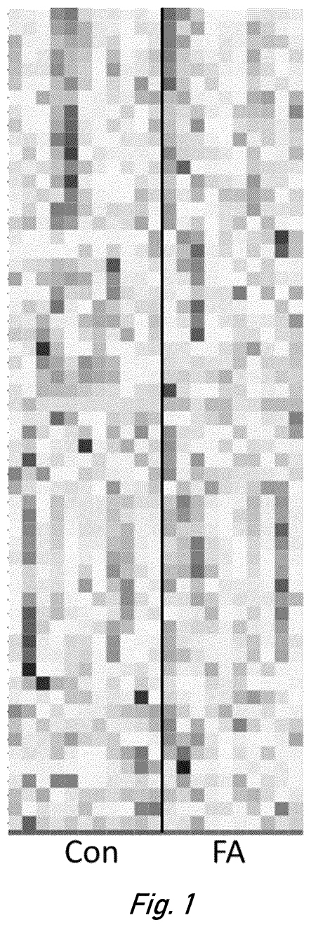

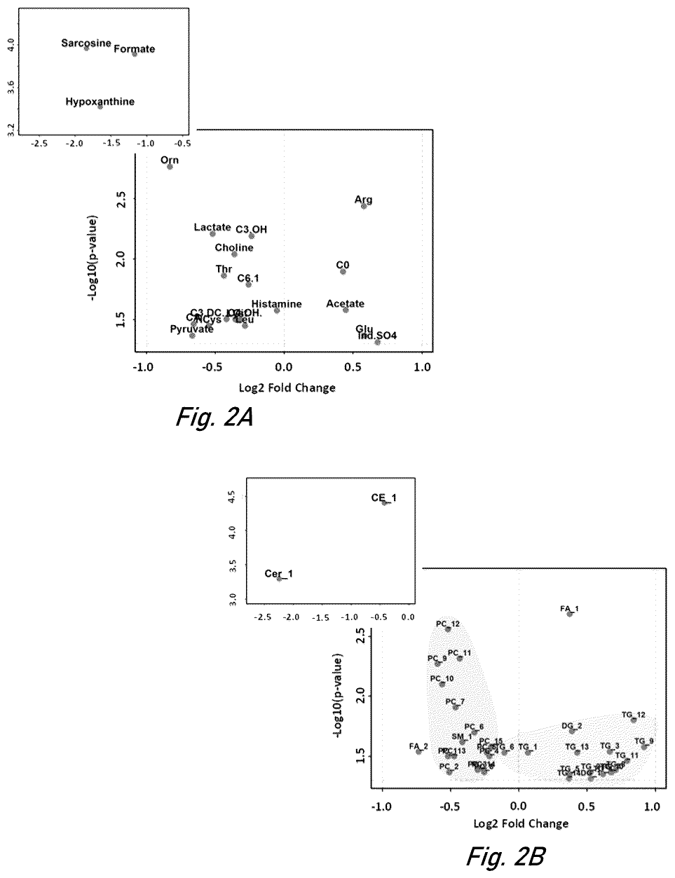

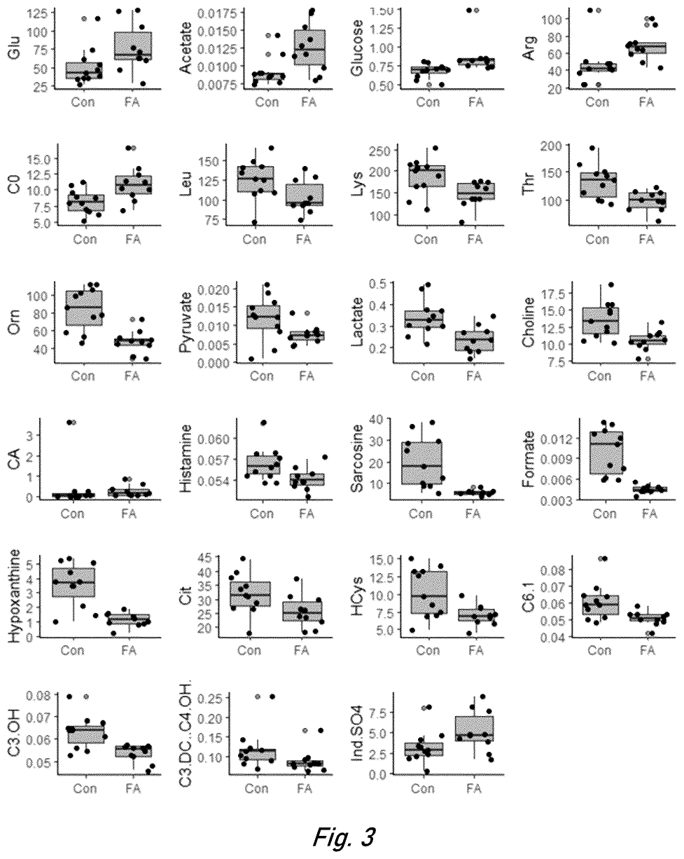

The combined approach described herein yielded quantitative measurements of 540 metabolites and found 59 unique metabolites (55 from MS and 4 from NMR) that were significantly different between the control and FA groups. A heatmap of the metabolites is shown in FIG. 1 revealing distinct metabolite patterns for the Con compared with the FA. To better represent the magnitude and significance of the specific changes, a set of volcano plots are shown in FIG. 2. To reduce crowding in the plots, the dataset was divided into the 23 hydrophilic metabolites and the 36 lipid-related metabolites. FIG. 2A shows the hydrophilic metabolites. The main plot shows metabolites with both positive and negative fold changes while the expansion shows that the three most significantly altered metabolites in terms of both fold change and significance are sarcosine, hypoxanthine and formate. Table 3 lists the log₂-fold changes and Wilcoxon p-values for these metabolites. The individual metabolite differences are shown as boxplots in FIG. 3.

TABLE 3

| Tables Metabolite | Tables log₂FC | Tables Wilcoxon p-value |
|---|---|---|
| Acetate | 0.44720 | 0.02640 |
| Formate | −1.17012 | 0.00012 |
| Glucose | 0.33957 | 0.00055 |
| Lactate | −0.51820 | 0.00619 |
| Pyruvate | −0.66791 | 0.04296 |
| Carnitine (C0) | 0.42751 | 0.01272 |
| Malonylcarnitine | −0.41935 | 0.03146 |
| (C3•DC . . . D4•OH)* | | |
| Hydroxypropionly- | −0.23666 | 0.00646 |
| carnitine (C3•OH) | | |
| Hexenoylcarnitine (C6.1) | −0.25752 | 0.01630 |
| Arginine | 0.57698 | 0.00365 |
| Glutamine | 0.58333 | 0.04296 |
| Leucine | −0.28398 | 0.03570 |
| Lysine | −0.35375 | 0.03162 |
| Threonine | −0.43715 | 0.01369 |
| Citrulline (Cit) | −0.31866 | 0.03168 |
| Homocysteine (Hcys) | −0.54804 | 0.03570 |
| Ornithine (Orn) | −0.82846 | 0.00172 |
| Sarcosine | −1.84145 | 0.00011 |
| Cholic acid (CA) | −0.65853 | 0.03446 |
| Histamine | −0.05381 | 0.02662 |
| Indoxylsulfate (Ind•SO₄) | 0.67727 | 0.04857 |
| Hypoxanthine | −1.65041 | 0.00038 |
| Choline | −0.36151 | 0.00913 |

For Table 3, P-value and Log₂ Fold Changes from Volcano plot in FIG. 2A. P-values were determined using the Wilcoxon rank sum test. *Malonylcarnitine=C3.DC.C4.OH, can also be assigned as the isobaric compound hydroxybutyrylcarnitine

FIG. 2B shows the volcano plot for the 36 lipid-related species. The expansion shows the two most significantly altered lipids are a cholesterol ester having an unsaturated 20 carbon fatty acid chain and a dihydroceramide with 18 and 22 carbon unsaturated fatty acid chains. The main plot shows that most of the lipids are triglycerides (TG) or phosphocholines (PC). To minimize overlap of the labels, the TGs and PCs were numbered. The fold change and significance values along with the full metabolite names are included in Table 4. With one exception (TG_6), all of the triglycerides are elevated in the FA patients while all of the phosphocholines are decreased.

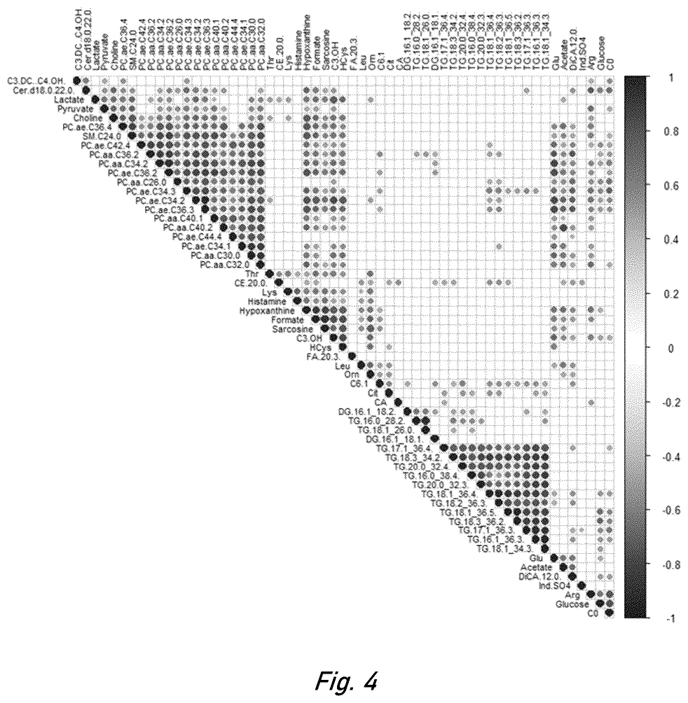

To discern patterns among the altered metabolites, a Pearson correlation matrix was calculated to identify clusters of correlated metabolites. FIG. 4 shows a correlogram where the correlations that meet a p-value significance threshold of 0.05 are shown with positive correlations in blue and negative correlation in red. This plot makes clear that the TG and PC lipids have strong inter-correlations. Near the center is a set of metabolites that includes hypoxanthine, formate, sarcosine, hydroxypropionylcarnitine (C3.OH) and homocysteine. These metabolites show significant positive correlations to many of the PCs along with negative correlations to a cluster of metabolites at the bottom of the chart which includes acetate, dodecanedioc acid (DiCA.12.0), indoxylsulfate (Ind.SO4), arginine, glucose and carnitine (C0). This latter cluster also shows significant negative correlations with many of the PCs.

TABLE 4

| ID | Metab | log₂FC | Wilcoxon p-value |
|---|---|---|---|
| FA 1 | FA(12:0) | 0.37114 | 0.00206 |
| CE 1 | CE(20:0) | −0.42446 | 0.00004 |
| DG_1 | DG.16.1 18.1 | 0.52848 | 0.04857 |
| DG_2 | DG (16:1, 18:2) | 0.38836 | 0.01966 |
| Cer_1 | Cer.d(18:0, 22:0) | −2.24324 | 0.00051 |
| FA_2 | FA(20.3) | −0.73330 | 0.02899 |
| PC_1 | PCaa(C26:0) | −0.51879 | 0.03168 |
| PC_2 | PCaa(C30:0) | −0.50864 | 0.04296 |
| PC_3 | PCaa(C32:0) | −0.30077 | 0.04101 |
| PC_4 | PCaa(C34:2) | −0.21464 | 0.03162 |
| PC 5 | PCaa(C36:2) | −0.23406 | 0.02948 |
| PC 6 | PCaa (C40:1) | −0.32490 | 0.02010 |
| PC_7 | PCaa(C40:2) | −0.46364 | 0.01240 |
| PC_8 | PCae (C34:1) | −0.25320 | 0.04296 |
| PC_9 | PCae(C34:2) | −0.59460 | 0.00537 |
| PC_10 | PCae (C34:3) | −0.56231 | 0.00795 |
| PC 11 | PCae (C36:2) | −0.43202 | 0.00484 |
| PC_12 | PCae (C36:3) | −0.51869 | 0.00276 |
| PC_13 | PCae (C36:4) | −0.47479 | 0.03168 |
| PC 14 | PCae (C42:4) | −0.25932 | 0.04107 |
| PC_15 | PCae (C44:4) | −0.20419 | 0.02649 |
| SM_1 | SM(C24:0) | −0.41341 | 0.02419 |
| TG_1 | TG(16:0, 28:2) | 0.06762 | 0.02948 |
| TG_2 | TG(16:0, 38:4) | 0.52561 | 0.04296 |
| TG 3 | TG(16:1, 36:3) | 0.66728 | 0.02893 |
| TG_4 | TG(17:1, 36:3) | 0.61589 | 0.04469 |
| TG_5 | TG(17:1, 36:4) | 0.37195 | 0.04469 |
| TG_6 | TG(18:1, 26:0) | −0.10452 | 0.02948 |
| TG_7 | TG(18:1, 34:3) | 0.67327 | 0.04296 |
| TG_8 | TG(18:1, 36:4) | 0.70956 | 0.04107 |
| TG_9 | TG(18:1, 36:5) | 0.91736 | 0.02645 |
| TG_10 | TG(18:2, 36:3) | 0.68107 | 0.04296 |
| TG 11 | TG(18:3, 34:2) | 0.79391 | 0.03458 |
| TG_12 | TG(18:3, 36:2) | 0.84173 | 0.01587 |
| TG_13 | TG(20:0, 32:3) | 0.42618 | 0.02948 |
| TG 14 | TG(20:0, 32:4) | 0.36786 | 0.04857 |

For purposes of Table 4, Metabolite Names, p-value and Log₂ Fold Changes from Volcano plot in FIG. 2A. FA, fatty acid; CE, cholesterol ester, DG, diglyceride; Cer.d, dihydroceramide; PC, phosphocholine; SM, sphingomyelin; TG, triglyceride. For the PCs, the aa indicates that both fatty acids are linked with an ester bond, while the ae designation indicates that one has an ester linkage and the other has an ether linkage.

### Network Community Analysis Identifies Altered One-Carbon Metabolism

In order to further understand the patterns of metabolite changes, a correlation-based network community analysis was carried out using the Girvan-Newman algorithm. In this approach, the metabolites are the network nodes and the network edges (connections between nodes) are the Pearson correlations. The algorithm detects communities by iteratively removing edges from the graph to break it down into smaller, highly connected pieces i.e., communities. In the context of metabolomics, this approach is used to reveal clusters of metabolites that are highly correlated and thus can represent specific pathways. Note that not all of the significantly altered metabolites are shown in the network community diagram as the algorithm did not find sufficient connections for some metabolites to be included in a community.

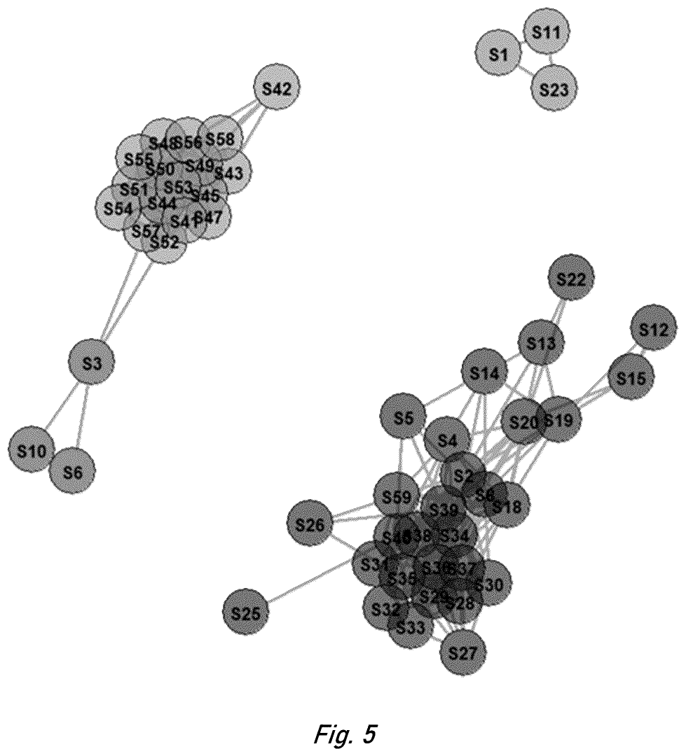

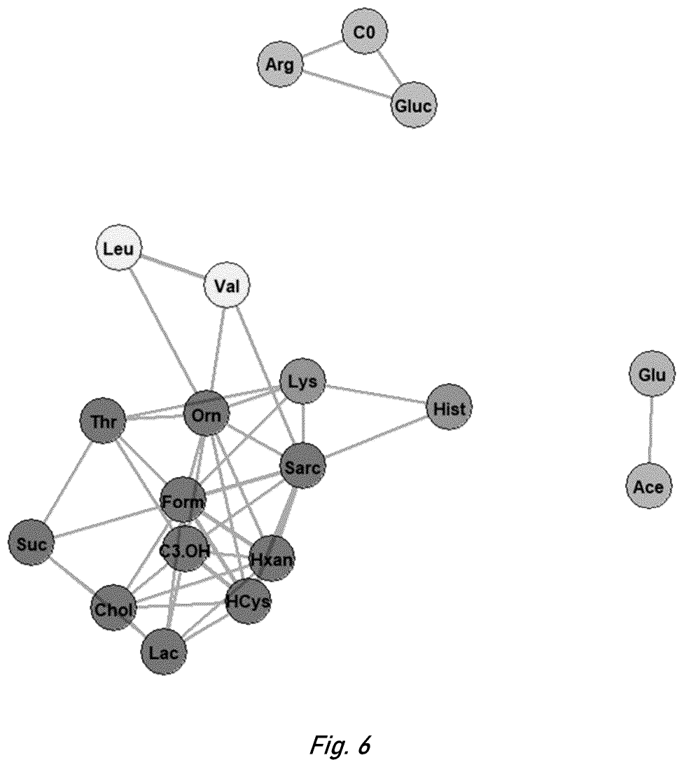

The network community analysis diagram generated using all 59 of the significant metabolites is shown in FIG. 5. The algorithm detected four distinct communities whose members are listed in Table 5. It is clear that that the second community (blue) is dominated by PCs and the fourth community (green) is dominated by TGs. To simplify the network, the lipids were excluded. FIG. 6 shows this reduced network community diagram which identified five communities. The community shown in green contains arginine, glucose and carnitine. The latter two metabolites play a major role in carbohydrate and fatty acid metabolism. The community shown in gray contains glutamate and acetate, which also have a relationship with energy metabolism. The community shown in red contains the amino acids lysine and histidine and the community in yellow contain contains the branched chain amino acids, leucine and valine. The largest community shown in blue forms a dense network of 10 metabolites. This community includes formate, sarcosine, hypoxanthine, choline, homocysteine, threonine, ornithine, lactate, hydroxypropionylcarnitine (C3.OH) and succinate. The first five of these metabolites suggest a connection to one-carbon metabolism.

TABLE 5
Network Community Membership for
all metabolites shown in FIG. 5

| ID | Community | Metabolite |
|---|---|---|
| S1 | 1 | Acetate |
| S11 | 1 | Glutamine |
| S23 | 1 | DiCA (12:0) |
| S12 | 2 | Leucine |
| S13 | 2 | Lysine |
| S14 | 2 | Threonine |
| S15 | 2 | Valine |
| S18 | 2 | Homocysteine (Hcys) |
| S19 | 2 | Ornathine (Orn) |
| S2 | 2 | Formate |
| S20 | 2 | Sarcosine |
| S22 | 2 | Histamine |
| S25 | 2 | DG (14:0_18:1) |
| S26 | 2 | Cer(d18:0/22:0) |
| S27 | 2 | PC aa C26:0 |
| S28 | 2 | PC aa: C30:0 |
| S29 | 2 | PC aa C34:2 |
| S30 | 2 | PC aa C36:2 |
| S31 | 2 | PC aa C:38:0 |
| S32 | 2 | PC aa C40:1 |
| S33 | 2 | PC aa C40:2 |
| S34 | 2 | PC ae C34:2 |
| S35 | 2 | PC ae C34:3 |
| S36 | 2 | PC ae C36:2 |
| S37 | 2 | PC ae C36:3 |
| S38 | 2 | PC ae C36:4 |
| S39 | 2 | Hypoxanthine |
| S4 | 2 | Lac.MS |
| S40 | 2 | SM C24:0 |
| S5 | 2 | Suc |
| S59 | 2 | Choline |
| S8 | 2 | C3-OH |
| S10 | 3 | Arginine |
| S3 | 3 | Glucose |
| S6 | 3 | C0 |
| S41 | 4 | TG(16:0_36:5) |
| S42 | 4 | TG(16:0_37:3) |
| S43 | 4 | TG(16:0_38:4) |
| S44 | 4 | TG(16:1_36:3) |
| S45 | 4 | TG(17:1_36:3) |
| S47 | 4 | TG(18:1_34:2) |
| S48 | 4 | TG(18:1_34:3) |
| S49 | 4 | TG(18:1_34:4) |
| S50 | 4 | TG(18:1_36:3) |
| S51 | 4 | TG(18:1_36:4) |
| S52 | 4 | TG(18:1_36:5) |
| S53 | 4 | TG(18:2_36:2) |
| S54 | 4 | TG(18:2_36:3) |
| S55 | 4 | TG(18:3_34:2) |
| S56 | 4 | TG(18:3_36:1) |
| S57 | 4 | TG(18:3_36:2) |
| S58 | 4 | TG(20:2_34:2) |

Metabolite Profiles Provide Accurate Discrimination of FA Patients from Controls

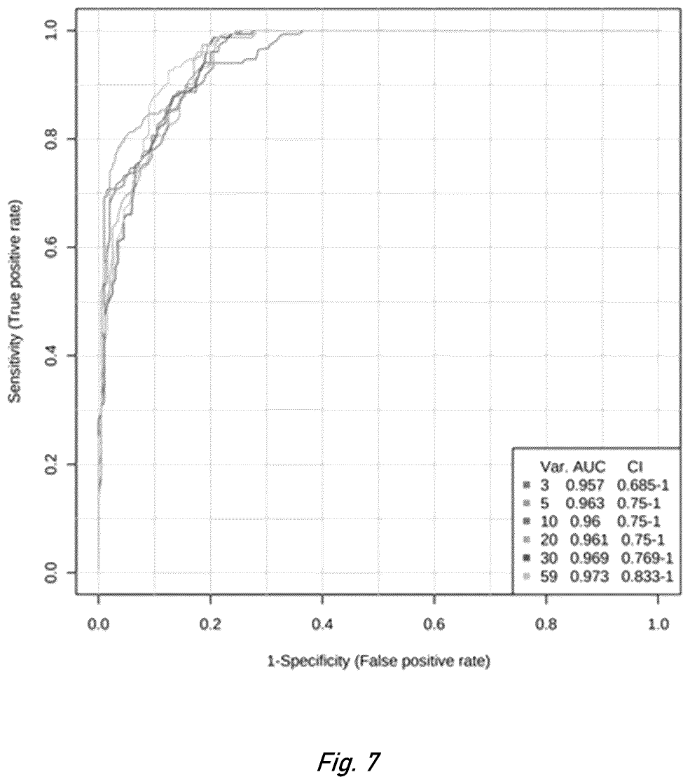

A primary goal of this study was to develop a metabolic biomarker panel that can be used to distinguish FA patients from controls. The composition of the panel elucidates the metabolic defects in FA and this panel can therefore be used to quickly monitor disease progression and the efficacy of therapeutic interventions. To evaluate the ability of this biomarker panel to distinguish between control and FA patients, receiver operating characteristic (ROC) analyses were carried out. Using Monte-Carlo cross validation along with classification and feature ranking using Random Forests, the ROC curves shown in FIG. 7 were generated. The curves include those made using the entire set of 59 metabolites along with various feature ranked subsets down to only three metabolites. The performance of the biomarker panel is evaluated by the areas under the curve (AUC) with a value of 1 being perfect classification and 0.5 being random. Each of the curves, including the one generated using only three metabolites provides excellent discrimination with a mean AUC value greater than 0.95. The confidence intervals also indicated excellent performance with the lowest value in the series being 0.685 when only three metabolites were used.

Beyond the metabolites associated with IC metabolism, the metabolic profiles also revealed other metabolites that appear to also play a role in the metabolic dysregulation in FA (FIG. 3). Pyruvate and lactate were reduced while glucose was increased in FA patients suggesting perturbations to glycolysis. The levels of carnitine (C0) were elevated in FA patients which suggests an effect on fatty acid oxidation. Interestingly, given the metabolic energy crisis from the mitochondrial dysfunction in FA, clinical trials of supplemental carnitine and creatine have been conducted to look for improved ATP production, but no significant results were observed.

Several other acylcarnitines were found to be decreased in FA patients including hydroxypropionylcarnitine (C3.OH, log₂FC=−0.237, p=0.0065), malonylcarnitine (C3-DC, log₂FC=−0.419, p=0.031; note that this metabolite could also be assigned as hydroxybutyrylcarnitine, C4-OH) and hexenoylcarnitine (C6:1) (Table 3). These hydroxylated and dicarboxylated acylcarnitines are associated with peroxisomal β-oxidation pathway which may be affected in FA.

Serum acetate levels were significantly increased in FA patients (log₂FC=0.447, p=0.026). Although acetate can come from numerous metabolic transformations, the major source of acetate in the blood is from gut microbial fermentation of dietary fibers. This significant increase may suggest a contribution from the gut microbiota in some of the metabolic derangements in FA.

### DISCUSSION

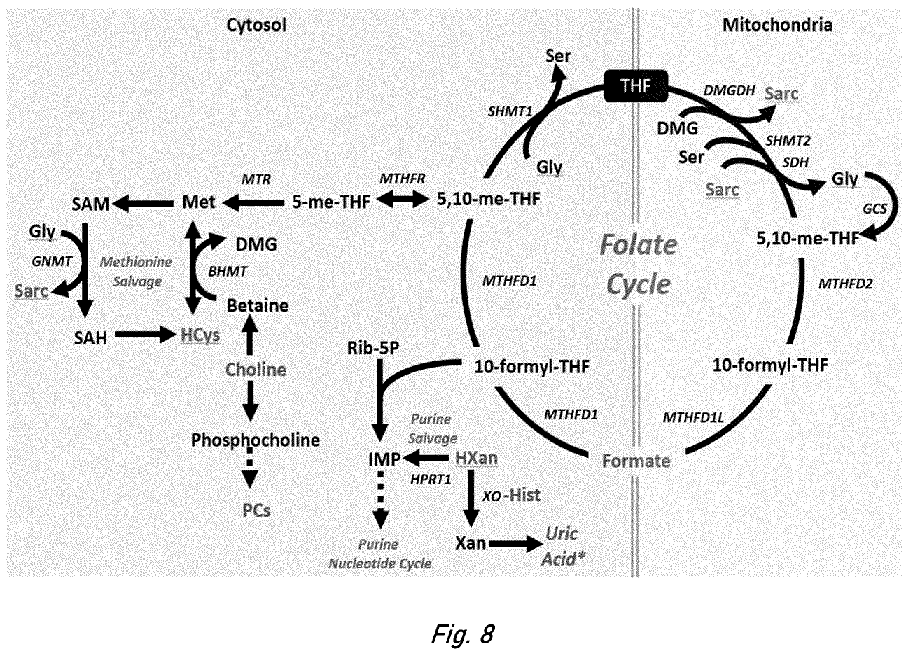

The pathway diagram shown in FIG. 8 shows the relationship between metabolic pathways and the metabolomics findings of this study. The major findings from the lipids were the significant decreases in phosphocholines and the increases in triglycerides. Elevated triglycerides are a consistent feature in FA patients. Even though none of the FA subjects had overt diabetes, some of this dyslipidemia could be the result of a pre-diabetic state given that the FA subjects had significantly higher serum glucose levels (FIG. 3). Other groups have previously reported that there is an increased prevalence of diabetes in FA patients. The observation of significantly reduced PCs, however, is a novel finding. Phosphatidylcholine synthesis is thought to be the largest 1C sink in adult mammals. As shown in FIG. 8, the reduced levels of choline (log₂FC=−0.362, p=0.009) could lead to the reduction in the PCs. Note that mammalian cells cannot synthesize choline and therefore it comes from the diet. The reduced levels therefore indicated increased usage. Another potential fate of choline is transformation to betaine to enable the synthesis of methionine from homocysteine. Maintaining the methionine levels and thus appropriate levels of S-adenosylmethionine (SAM) and S-adenosylhomocysteine (SAH) in the methionine salvage pathway is critical to preserving the methylating capacity of the cells. The reduced levels of homocysteine (log₂FC=−0.548, p=0.036) observed would also be consistent with dysregulation in the methionine salvage pathway.

The two metabolites with the most significance alterations are formate (log₂FC=−1.170, p=0.0001) and sarcosine (log₂FC=1.841, p=0.0001). As shown in FIG. 8, one-carbon units can be generated in the mitochondria in the folate cycle from the amino acids serine and glycine, as well as the choline metabolites, sarcosine and dimethylglycine. These reactions ultimately generate formate that can function as an inter-organ and inter-organelle shuttle of one-carbon units. The mitochondrial transformation of tetrahydrofolate (THF) to 5,10-methylene-THF can be coupled to the demethylation of serine or sarcosine to glycine via serine hydroxymethyltransferase (SHMT) or sarcosine dehydrogenase (SDH). Further transformation by methylenetetrahydrofolate dehydrogenase 2 (MTHFD2) yields 10-formyl-THF which is then converted back to THF with the release of formate by the action of methylenetetrahydrofolate dehydrogenase (MTHFDIL). The significant reduction in circulating formate found in FA patients is highly suggestive of dysregulated folate metabolism.

Other groups have previously reported that non-folate-dependent pathways also can generate formate, including the catabolism of threonine and kynurenine, peroxisomal β-oxidation of branched chain fatty acids, and cytochrome P-450 mediated demethylation and oxidation reactions. No differences in the levels of kynurenine were observed. The levels of threonine are indeed reduced in FA patients, which may indicate some contribution from this pathway (see, Table 3, Log₂FC=−0.437, p=0.014). The oxidation of branched chain fatty acids would be expected to be dependent primarily upon dietary sources of these fatty acids which would not likely be different in the FA and Con groups. Thus, it appears more plausible that the reduced formate levels are the result of folate cycle dysregulation.

Interestingly, the serine levels in the FA patients studied were not significantly altered. Serine is a major source of formate produced in mitochondria which is then released into the cytosol. As shown in FIG. 8, in the cytosol, the transformation of 5,10-methylene-THF back to THF is coupled to the resynthesis of serine from glycine via serine hydroxymethyltransferase 1 (SHMT2). The ability of the folate cycle to consume serine in the mitochondria and resynthesize it in the cytosol may play a role in the maintenance of serine levels, even under the conditions of a dysregulation of 1C metabolism.

The reduced levels of sarcosine also suggest perturbations in 1C metabolism. Sarcosine can be derived from the catabolism of choline which is coupled to the remethylation of homocysteine to methionine. In this process, choline is transformed to betaine aldehyde via choline dehydrogenase. This is followed by the action of aldehyde dehydrogenase yielding betaine. Betaine homocysteine-S-methyltransferase (BHMT) converts betaine and homocysteine to methionine and dimethylglycine. Finally, dimethylglycine is converted to sarcosine via dimethylglycine dehydrogenase. Sarcosine can then donate its methyl group to THF forming 5,10-methylene THF. Reduced levels of sarcosine can therefore lead to a reduction in folate 1C units.

Interestingly, dimethylglycine dehydrogenase (DMGDH), as well as sarcosine dehydrogenase (SDH), are both coupled to the electron transfer flavoproteins (ETF). ETFs are a group of Fe/S containing proteins that are electron acceptors of several dehydrogenases and feed electrons to ETF:ubiquinone oxidoreductase (ETF:QO) complex in the electron transport chain. Thus, dysregulation of DMGDH due to impaired Fe/S biogenesis may be a factor in the reduced sarcosine levels. Sarcosine can also be generated by the transformation of methionine derived SAM to SAH which is coupled to glycine-N-methyltransferase GNMT) which donates a methyl group to glycine forming sarcosine.

As there is no established essential function for sarcosine, the function of GNMT may not be to generate sarcosine, but to regulate the levels of SAM and SAH. The overall SAM/SAH ratio is sometimes considered to be an index of the methylating capacity of the cell. The dysregulated sarcosine levels could lead to wide ranging dysregulation in cellular methylation reactions.

Purine nucleotide synthesis is connected to the folate cycle through the reaction of 10-formyl-THF with ribose-5-phosphate to form inosine monophosphate (IMP), which goes on to form other purine nucleotides; see FIG. 8. Given a potential deficit in the production of 10-formyl-THF by a dysregulated folate cycle, purine nucleotide synthesis may be deficient as well. Another potential resource for purine nucleotide synthesis is through the salvage pathway involving the conversion of hypoxanthine (HXan) to IMP via hypoxanthine-guanine phosphoribosyltransferase (HPRT1). The reduced levels of HXan (see Table 3, log₂FC=−1.650, p=0.0004) would be consistent with an attempt of the purine salvage pathway to support purine nucleotide synthesis during a deficit of 10-formyl-THF.

### Mitochondrial Dysfunction as a Driver of Dysregulation in One-Carbon Metabolism.

A strong connection between mitochondrial functioning and 1C metabolism has been shown in several recent studies. In an elegant study by Bao et al., cells were generated with a dominant negative mutant of DNA polymerase gamma, thereby stopping replication of mtDNA and leading to the depletion of components of oxidative phosphorylation. Bao et al. demonstrated that lesioning the respiratory chain impaired mitochondrial production of formate from serine and that in some cells, respiratory chain inhibition leads to growth defects upon withdrawal of serine that were rescued with purine or formate supplementation.

In a study of cultured cancer cells, Meisner et al., showed that most of the serine derived one carbon units are released from the cells as formate and depend upon reverse 10-formyl-THF activity. Formate release was coupled to mitochondrial complex I activity and it was found that both serine starvation and complex I inhibition led to reduced formate synthesis.

Other variations or embodiments will be apparent to a person of ordinary skill in the art from the above description. Thus, the foregoing embodiments are not to be construed as limiting the scope of the claimed invention. All references disclosed are expressly incorporated by reference in in their entirety.

### REFERENCES

Pandolfo, M. (2009) Friedreich ataxia: the clinical picture. J Neurol, 256 Suppl 1, 3-8.
Reddy, P. L. and Grewal, R. P. (2007) Friedreich's ataxia: a clinical and genetic analysis. Clinical neurology and neurosurgery, 109, 200-202.
De Biase, I., Rasmussen, A., Endres, D., Al-Mahdawi, S., Monticelli, A., Cocozza, S., Pook, M. and Bidichandani, S. I. (2007) Progressive GAA expansions in dorsal root ganglia of Friedreich's ataxia patients. Ann Neurol, 61, 55-60.
Tsou, A. Y., Paulsen, E. K., Lagedrost, S. J., Perlman, S. L., Mathews, K. D., Wilmot, G. R., Ravina, B., Koeppen, A. H. and Lynch, D. R. (2011) Mortality in friedreich ataxia. J Neurol Sci., 307, 46-49.
Hewer, R. L. (1968) Study of fatal cases of Friedreich's ataxia. BMJ (Clinical research ed, 3, 649-652.
Schols, L., Amoiridis, G., Przuntek, H., Frank, G., Epplen, J. T. and Epplen, C. (1997) Friedreich's ataxia: revision of the phenotype according to molecular genetics. Brain, 120, 2131-2140.
Filla, A., De Michele, G., Marconi, R., Bucci, L., Carillo, C., Castellano, A. E., Iorio, L., Kniahynicki, C., Rossi, F. and Campanella, G. (1992) Prevalence of hereditary ataxias and spastic paraplegias in Molise, a region of Italy. J Neurol, 239, 351-353.
Romeo, G., Menozzi, P., Ferlini, A., Fadda, S., Di Donato, S., Uziel, G., Lucci, B., Capodaglio, L., Filla, A. and Campanella, G. (1983) Incidence of Friedreich ataxia in Italy estimated from consanguineous marriages. Am J Hum Genet, 35, 523-529.
Skre, H. (1975) Friedreich's ataxia in Western Norway. Clin Genet, 7, 287-298.
Epplen, C., Epplen, J. T., Frank, G., Miterski, B., Santos, E. J. and Schols, L. (1997) Differential stability of the (GAA) n tract in the Friedreich ataxia (STM7) gene. Hum Genet, 99, 834-836.
Campuzano, V., Montermini, L., Molto, M. D., Pianese, L., Cossee, M., Cavalcanti, F., Monros, E., Rodius, F., Duclos, F., Monticelli, A. et al. (1996) Friedreich's ataxia: autosomal recessive disease caused by an intronic GAA triplet repeat expansion. Science, 271, 1423-1427.
Montermini, L., Richter, A., Morgan, K., Justice, C. M., Julien, D., Castellotti, B., Mercier, J., Poirier, J., Capozzoli, F., Bouchard, J. P. et al. (1997) Phenotypic variability in Friedreich ataxia: role of the associated GAA triplet repeat expansion. Ann Neurol, 41, 675-682.
Filla, A., De Michele, G., Cavalcanti, F., Pianese, L., Monticelli, A., Campanella, G. and Cocozza, S. (1996) The relationship between trinucleotide (GAA) repeat length and clinical features in Friedreich ataxia. Am J Hum Genet, 59, 554-560.
Pastore, A. and Puccio, H. (2013) Frataxin: a protein in search for a function. J Neurochem, 126 Suppl 1, 43-52.
Fox, N. G., Yu, X., Feng, X., Bailey, H. J., Martelli, A., Nabhan, J. F., Strain-Damerell, C., Bulawa, C., Yue, W. W. and Han, S. (2019) Structure of the human frataxin-bound iron-sulfur cluster assembly complex provides insight into its activation mechanism. Nat Commun, 10, 2210.
Wagner, G. R., Pride, P. M., Babbey, C. M. and Payne, R. M. (2012) Friedreich's ataxia reveals a mechanism for coordinate regulation of oxidative metabolism via feedback inhibition of the SIRT3 deacetylase. Human Molecular Genetics, 21, 2688-2697.
Martin, A. S., Abraham, D. M., Hershberger, K. A., Bhatt, D. P., Mao, L., Cui, H., Liu, J.,
Liu, X., Muehlbauer, M. J., Grimsrud, P. A. et al. (2017) Nicotinamide mononucleotide requires SIRT3 to improve cardiac function and bioenergetics in a Friedreich's ataxia cardiomyopathy model. JCI Insight, 2.
Haugen, A. C., Di Prospero, N. A., Parker, J. S., Fannin, R. D., Chou, J., Meyer, J. N., Halweg, C., Collins, J. B., Durr, A., Fischbeck, K. et al. (2010) Altered gene expression and DNA damage in peripheral blood cells from Friedreich's ataxia patients: cellular model of pathology. PLoS Genet, 6, e1000812.
Wagner, G. R. and Payne, R. M. (2013) Widespread and enzyme-independent Nepsilonacetylation and Nepsilon-succinylation of proteins in the chemical conditions of the mitochondrial matrix. The Journal of biological chemistry, 288, 29036-29045.
Stram, A. R., Wagner, G. R., Fogler, B. D., Pride, P. M., Hirschey, M. D. and Payne, R. M. (2017) Progressive mitochondrial protein lysine acetylation and heart failure in a model of Friedreich's ataxia cardiomyopathy. PLoS One, 12, e0178354.
Reetz, K., Dogan, I., Hilgers, R. D., Giunti, P., Parkinson, M. H., Mariotti, C., Nanetti, L., Durr, A., Ewenczyk, C., Boesch, S. et al. (2021) Progression characteristics of the European Friedreich's Ataxia Consortium for Translational Studies (EFACTS): a 4-year cohort study. Lancet Neurol, 20, 362-372.
Clish, C. B. (2015) Metabolomics: an emerging but powerful tool for precision medicine. Cold Spring Harb Mol Case Stud, 1, a000588.
Newman, M. E. and Girvan, M. (2004) Finding and evaluating community structure in networks. Phys Rev E Stat Nonlin Soft Matter Phys, 69, 026113.
Hayashi, G., Shen, Y., Pedersen, T. L., Newman, J. W., Pook, M. and Cortopassi, G. (2014) Frataxin deficiency increases cyclooxygenase 2 and prostaglandins in cell and animal models of Friedreich's ataxia. Hum Mol Genet, 23, 6838-6847.
Zhao, H., Li, H., Hao, S., Chen, J., Wu, J., Song, C., Zhang, M., Qiao, T. and Li, K. (2017) Peptide SS-31 upregulates frataxin expression and improves the quality of mitochondria: implications in the treatment of Friedreich ataxia. Sci Rep, 7, 9840.
Wong, A., Yang, J., Cavadini, P., Gellera, C., Lonnerdal, B., Taroni, F. and Cortopassi, G. (1999) The Friedreich's ataxia mutation confers cellular sensitivity to oxidant stress which is rescued by chelators of iron and calcium and inhibitors of apoptosis. Hum. Mol. Genet., 8, 425-430.
Rustin, P., Bonnet, D., Rotig, A., Munnich, A. and Sidi, D. (2004) Idebenone treatment in Friedreich patients: one-year-long randomized placebo-controlled trial. Neurology, 62, 524-525; author reply 525; discussion 525.
Pandolfo, M., Arpa, J., Delatycki, M. B., Le Quan Sang, K. H., Mariotti, C., Munnich, A., Sanz-Gallego, I., Tai, G., Tarnopolsky, M. A., Taroni, F. et al. (2014) Deferiprone in Friedreich ataxia: a 6-month randomized controlled trial. Ann Neurol, 76, 509-521.
Hart, P. E., Lodi, R., Rajagopalan, B., Bradley, J. L., Crilley, J. G., Turner, C., Blamire, A. M., Manners, D., Styles, P., Schapira, A. H. et al. (2005) Antioxidant treatment of patients with Friedreich ataxia: four-year follow-up. Arch Neurol, 62, 621-626.
Burk, K. (2017) Friedreich Ataxia: current status and future prospects. Cerebellum Ataxias, 4, 4.
Huang, Y. S., Nestruck, A. C., Barbeau, A., Bouchard, J. P. and Davignon, J. (1978) Plasma lipids and lipoproteins in Friedreich's ataxia and familial spastic ataxia—evidence for an abnormal composition of high density lipoproteins. Can J Neurol Sci, 5, 149-156.
Wang, Q., Guo, L., Strawser, C. J., Hauser, L. A., Hwang, W. T., Snyder, N. W., Lynch, D. R., Mesaros, C. and Blair, I. A. (2018) Low apolipoprotein A-I levels in Friedreich's ataxia and in frataxin-deficient cells: Implications for therapy. PLoS One, 13, e0192779.
Cnop, M., Mulder, H. and Igoillo-Esteve, M. (2013) Diabetes in Friedreich ataxia. J Neurochem, 126 Suppl 1, 94-102.
Finocchiaro, G., Baio, G., Micossi, P., Pozza, G. and di Donato, S. (1988) Glucose metabolism alterations in Friedreich's ataxia. Neurology, 38, 1292-1296.
Stead, L. M., Brosnan, J. T., Brosnan, M. E., Vance, D. E. and Jacobs, R. L. (2006) Is it time to reevaluate methyl balance in humans? Am J Clin Nutr, 83, 5-10.
Ducker, G. S. and Rabinowitz, J. D. (2017) One-Carbon Metabolism in Health and Disease. Cell Metab, 25, 27-42.
Henriques, B. J., Katrine Jentoft Olsen, R., Gomes, C. M. and Bross, P. (2021) Electron transfer flavoprotein and its role in mitochondrial energy metabolism in health and disease. Gene, 776, 145407.
Luka, Z., Mudd, S. H. and Wagner, C. (2009) Glycine N-methyltransferase and regulation of S-adenosylmethionine levels. J Biol Chem, 284, 22507-22511.
Schirinzi, T., Vasco, G., Zanni, G., Petrillo, S., Piemonte, F., Castelli, E. and Bertini, E. S. (2018) Serum uric acid in Friedreich Ataxia. Clin Biochem, 54, 139-141.
Bao, X. R., Ong, S. E., Goldberger, O., Peng, J., Sharma, R., Thompson, D. A., Vafai, S. B., Cox, A. G., Marutani, E., Ichinose, F. et al. (2016) Mitochondrial dysfunction remodels one carbon metabolism in human cells. eLife, 5.
Meiser, J., Tumanov, S., Maddocks, O., Labuschagne, C. F., Athineos, D., Van Den Broek, N., Mackay, G. M., Gottlieb, E., Blyth, K., Vousden, K. et al. (2016) Serine one-carbon catabolism with formate overflow. Sci Adv, 2, e1601273.
Beckonert, O., Keun, H. C., Ebbels, T. M., Bundy, J., Holmes, E., Lindon, J. C. and Nicholson, J. K. (2007) Metabolic profiling, metabolomic and metabonomic procedures for NMR spectroscopy of urine, plasma, serum and tissue extracts. Nature protocols, 2, 2692-2703.
Choudhury, R., Beezley, J., Davis, B., Tomeck, J., Gratzl, S., Golzarri-Arroyo, L., Wan, J., Raftery, D., Baumes, J. and O'Connell, T. M. (2020) Viime: Visualization and Integration of Metabolomics Experiments. The Journal of Open Source Software, 5, 2410-2423.
R Core Team, (2017), R: A language and environment for statistical computing. R Foundation for Statistical Computing, Vienna, Austria. URL https://www.r-project.org/.
Chong, J., Soufan, O., Li, C., Caraus, I., Li, S., Bourque, G., Wishart, D. S. and Xia, J. (2018) MetaboAnalyst 4.0: towards more transparent and integrative metabolomics analysis. Nucleic Acids Res, 46, W486-W494.
Muraoka, S. (1963) Studies on xanthine oxidase: II. Mechanism of substrate inhibition and its reversal by histamine. Biochimica et Biophysica Acta (BBA)—Specialized Section on Enzymological Subjects, 73, 27-38.
Schols, L., Zange, J., Abele, M., Schillings, M., Skipka, G., Kuntz-Hehner, S., van Beekvelt, M. C., Colier, W. N., Muller, K., Klockgether, T. et al. (2005) L-carnitine and creatine in Friedreich's ataxia. A randomized, placebo-controlled crossover trial. J Neural Transm, 112, 789-796.
Koh, A., De Vadder, F., Kovatcheva-Datchary, P. and Backhed, F. (2016) From Dietary Fiber to Host Physiology: Short-Chain Fatty Acids as Key Bacterial Metabolites. Cell, 165, 1332-1345.

1. A method to assess a subject having Friedreich's ataxia, the method comprising:
determining a concentration level of a panel of at least two validated low molecular weight metabolic biomarkers in a biological sample from the subject;
identifying a difference between the determined concentration level of the at least two metabolic biomarkers and a reference concentration level of the at least two metabolic biomarkers; and,
assessing the subject based on the identified difference.

2. The method according to claim 1, where the assessing step includes determining a stage of progression of Friedreich's ataxia in the subject.

3. The method according to claim 1, where the assessing step includes differentially and/or comparatively diagnosing between symptomatic Friedreich's ataxia and late-symptomatic Friedreich's ataxia.

4. The method according to claim 1, where the subject is undergoing a treatment for Friedreich's ataxia and the assessing step includes determining whether to continue treatment, to discontinue treatment, or to alter treatment based on presence, absence, or the concentration level of the at least two metabolic biomarkers in the panel.

5. The method according to claim 1, where the subject is undergoing a treatment for Friedreich's ataxia and the assessing step includes monitoring an effect of the treatment on the subject.

6. The method according to claim 1, further comprising comparing the identified differences in a biological sample taken from a subject at two or more points in time.

7. The method according to claim 1, further comprising comparing the identified differences in a biological sample taken from a subject at two or more points in time, where a change in the identified differences toward a phenotype profile, is interpreted as a progression toward the phenotype.

8. The method according to claim 1, further comprising comparing the identified differences in a biological sample taken from a subject before and after a treatment or during a course of treatment.

9. The method according to claim 1, further comprising comparing the identified differences in a biological sample taken from a subject before and after a treatment or during a course of treatment, where a change in biomarker profile over time toward an asymptomatic profile or to a stable profile is interpreted as efficacy.

10. The method according to claim 1, further comprising: (i) providing a recommended treatment; and, (ii) administering the treatment to the subject.

11. (canceled)

12. (canceled)

13. A diagnostic kit for assessing a subject for an attribute of Friedreich's ataxia, where the attribute of Friedreich's ataxia is associated with a low molecular weight metabolic biomarker profile, the kit comprising: a container comprising a panel of at least two validated low molecular weight metabolic biomarker internal standards having a purity greater than 98.0%, where the container is configured to receive a biological sample from the subject and to be sealed with a sealing member after receiving the biological sample.

14. A method to identify a candidate low molecular weight metabolic biomarker panel that differentially and/or comparatively diagnoses a subject having Friedreich's ataxia, the method comprising:
obtaining a first biological sample from a first subject having a first phenotype associated with Friedreich's ataxia and a second biological sample from a second subject having a second phenotype associated with Friedreich's ataxia;
determining a concentration level of at least two low molecular weight metabolic biomarkers in the first biological sample, the second biological sample, and a reference concentration level of the at least two metabolic biomarkers; and,
identifying two or more validated low molecular weight metabolic biomarkers that are present in a statistically significant, different level in the first and second biological samples, where the different level is characterized by an area under the receiver operator characteristic (ROC) curve (AUC) ranging from 0.60 to 1.00.

15. (canceled)

16. (canceled)

17. (canceled)

18. (canceled)
